SEQUENCE LISTING (44 sequences)

SEQ ID NO: 1 - protein, 8 aa, Artificial Sequence
  Linker Sequence
GGGSGGGS

SEQ ID NO: 2 - protein, 10 aa, Artificial Sequence
  Linker Sequence
GGGGSGGGGS

SEQ ID NO: 3 - protein, 12 aa, Artificial Sequence
  Linker Sequence
GGGSGGGSGGGS

SEQ ID NO: 4 - protein, 15 aa, Artificial Sequence
  Linker Sequence
GGGGSGGGGSGGGGS

SEQ ID NO: 5 - protein, 16 aa, Artificial Sequence
  Linker Sequence
GGGSGGGSGGGSGGGS

SEQ ID NO: 6 - protein, 20 aa, Artificial Sequence
  Linker Sequence
GGGGSGGGGSGGGGSGGGGS

SEQ ID NO: 7 - protein, 20 aa, Artificial Sequence
  Linker Sequence
GGGSGGGSGGGSGGGSGGGS

SEQ ID NO: 8 - protein, 25 aa, Artificial Sequence
  Linker Sequence
GGGGSGGGGSGGGGSGGGGSGGGGS

SEQ ID NO: 9 - protein, 24 aa, Artificial Sequence
  Linker Sequence
GGGSGGGSGGGSGGGSGGGSGGGS

SEQ ID NO: 10 - protein, 30 aa, Artificial Sequence
  Linker Sequence
GGGGSGGGGSGGGGSGGGGSGGGGSGGGGS

SEQ ID NO: 11 - protein, 28 aa, Artificial Sequence
  Linker Sequence
GSADDAKKDAAKKDAAKKDDAKKDDAGS

SEQ ID NO: 12 - protein, 32 aa, Artificial Sequence
  Linker Sequence
GSADDAKKDAAKKDAAKKDDAKKDDAKKDAGS

SEQ ID NO: 13 - protein, 8 aa, Artificial Sequence
  Linker Sequence
GGGQGGGQ

SEQ ID NO: 14 - protein, 10 aa, Artificial Sequence
  Linker Sequence
GGGGQGGGGQ

SEQ ID NO: 15 - protein, 12 aa, Artificial Sequence
  Linker Sequence
GGGQGGGQGGGQ

SEQ ID NO: 16 - protein, 15 aa, Artificial Sequence
  Linker Sequence
GGGGQGGGGQGGGGQ

SEQ ID NO: 17 - protein, 16 aa, Artificial Sequence
  Linker Sequence
GGGQGGGQGGGQGGGQ

SEQ ID NO: 18 - protein, 20 aa, Artificial Sequence
  Linker Sequence
GGGGQGGGGQGGGGQGGGGQ

SEQ ID NO: 19 - protein, 20 aa, Artificial Sequence
  Linker Sequence
GGGQGGGQGGGQGGGQGGGQ

SEQ ID NO: 20 - protein, 25 aa, Artificial Sequence
  Linker Sequence
GGGGQGGGGQGGGGQGGGGQGGGGQ

SEQ ID NO: 21 - protein, 24 aa, Artificial Sequence
  Linker Sequence
GGGQGGGQGGGQGGGQGGGQGGGQ

SEQ ID NO: 22 - protein, 30 aa, Artificial Sequence
  Linker Sequence
GGGGQGGGGQGGGGQGGGGQGGGGQGGGGQ

SEQ ID NO: 23 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 24 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 25 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 26 - protein, 106 aa, Homo sapiens
GQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 27 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 330 aa, Homo sapiens [sequence omitted]

SEQ ID NO: 29 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 22 aa, Homo sapiens
MDMRVPAQLLGLLLLWLRGARC

SEQ ID NO: 32 - protein, 19 aa, Homo sapiens
MAWALLLLTLLTQGTGSWA

SEQ ID NO: 33 - protein, 19 aa, Homo sapiens
MTCSPLLLTLLIHCTGSWA

SEQ ID NO: 34 - protein, 20 aa, Homo sapiens
MEAPAQLLFLLLLWLPDTTG

SEQ ID NO: 35 - protein, 19 aa, Homo sapiens
MEWTWRVLFLVAAATGAHS

SEQ ID NO: 36 - protein, 20 aa, Homo sapiens
METPAQLLFLLLLWLPDTTG

SEQ ID NO: 37 - protein, 19 aa, Homo sapiens
MKHLWFFLLLVAAPRWVLS

SEQ ID NO: 38 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 39 - protein, 19 aa, Homo sapiens
MEWSWVFLFFLSVTTGVHS

SEQ ID NO: 40 - protein, 12 aa, Artificial Sequence
  Linker Sequence
GGGGSGGGGSGG

SEQ ID NO: 41 - protein, 17 aa, Artificial Sequence
  Linker Sequence
GGGGSGGGGSGGGGSGG

SEQ ID NO: 42 - protein, 696 aa, Artificial Sequence
  scFv/Heavy Chain Fusion
DIVMTQTPLSLSVTPGQPASISCKSSQSLLYSDGKTYLYWFLQKPGQPPQLLIYEVSNRFSGVPDRFSGSGSGTDFTLKISRVEAEDVGVYYCMQSIQLPRTFGQGTKVEIKRGGGGSGGGGSGGQVHLVESGGGVVQPGRSLRLSCAASGFTFSFYGMHWVRQAPGKGLEWVAVIWYDGNNQYYADSVKGRFTISRDNSKNTLYLQMNSLRAEDTAVYYCARDGDYYYGMDVWGQGGGGGSGGGGSGGQVQLVQSGAEVKKPGASVKVSCKASGYTFTDYYLHWVRQAPGQGLEWMGWIHPNSGGTNYAQKFQGRVTMTRDTSISTAYMGLSSLRSDDTAVYYCARDGTSSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 43 - protein, 214 aa, Homo sapiens
DIQMTQSPSSVSASVGDRVTITCRASQGISRWLAWYQQKPGKAPKLLIYGASSLQSGVPSRFSGSGSGTDFTLTISSLQPEDFAIYYCQQANSFPFTFGPGTKVDIKGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 227 aa, Artificial Sequence
  Heavy Chain Fc Region
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

(19) United States
(12) Patent Application Publication
(10) Pub. No.: US 2023/0047631 A1
(43) Pub. Date: Feb. 16, 2023

(54) NOVEL MULTISPECIFIC ANTIBODY FORMAT

(71) Applicant: AMGEN INC., Thousand Oaks, CA (US)

(72) Inventors: Fernando GARCES, Studio City, CA (US); Zhulun WANG, Los Altos, CA (US)

(21) Appl. No.: 17/778,361

(22) PCT Filed: Nov. 18, 2020
(86) PCT No.: PCT/US2020/061124

§ 371 Date: May 19, 2022

Related U.S. Application Data
(60) Provisional application No. 62/937,729, filed on Nov. 19, 2019.

(51) Int. Cl. C07K 16/46

(52) U.S. Cl. CPC C07K 16/468; C07K 2317/31; C07K 2317/53; C07K 2317/524; C07K 2317/526; C07K 2317/622; C07K 2317/522; C07K 2317/55

(57) ABSTRACT

The present invention relates to novel multispecific antigen binding proteins that are capable of binding to multiple targets. Pharmaceutical compositions comprising the multispecific antigen binding proteins as well as methods for producing them are also disclosed.

### FIELD OF THE INVENTION

The present invention relates to multispecific antibodies, polynucleotides encoding multispecific antibodies, and methods of making multispecific antibodies.

The instant application contains an ASCII “txt” compliant sequence listing which serves as both the computer readable form (CRF) and the paper copy required by 37 C.F.R. Section 1.821(c) and 1.821(e), and is hereby incorporated by reference in its entirety. The name of the “txt” file created on Nov. 18, 2020, is: A-2536-WO-PCT_SEQ_LIST_20201118_ST25, and is 36.1 kb in size.

### BACKGROUND OF THE INVENTION

The clinical potential of multispecific antibodies (molecules that target multiple targets simultaneously) like bispecific and trispecific antibodies shows great promise for targeting complex diseases. However, the generation of those molecules displaying the desired activity presents great challenges. Here, we describe a new format that allows for fine tuning of the binding from the two antigen binding domains by tethering them via linker with different lengths and sequences in order to optimize antigen engagement (FIG. 1).

Although over 100 bispecific formats have been reported, often they fail to deliver the bispecific biology that is intended to. Such unfortunate outcome is often linked to the lack of knowledge regarding the spatial position of epitopes and respective therapeutic targets on the surface of the cell. Armed with that knowledge, this bispecific format can be tuned to meet the specific requirements for on-target activity while minimizing the off-target activity, which is critical to deliver efficacious and safe therapeutics.

### SUMMARY OF THE INVENTION

In one aspect the present invention is directed to a multispecific antibody construct comprising:

a) a first polypeptide comprising an antibody Fc region, the antibody Fc region comprising a first hinge region, a first CH2 region, and a first CH3 region;

b) a second polypeptide comprising an antibody heavy chain construct, the antibody heavy chain construct comprising

i) a scFv, the scFv comprising

1) a first VH and a first VL,

wherein the first VH and the first VL associate to form a first antigen binding domain, and

2) a first linker peptide that connects the first VH and first VL; and

ii) an antibody heavy chain, the antibody heavy chain comprising a second VH, a second CH1 region, a second hinge region, a second CH2 region, and a second CH3 region;

wherein the scFv is attached at its C-terminus to the N-terminus of the second VH region of the antibody heavy chain;

c) a third polypeptide comprising an antibody light chain comprising a second VL and a CL,

wherein the second VH of the antibody heavy chain and the second VL of the antibody light chain associate to form a second antigen binding domain.

In another aspect, the present invention is directed to a multispecific antibody construct comprising:

a) a first polypeptide comprising an antibody Fc region, the antibody Fc region comprising a first hinge region, a first CH2 region, and first CH3 regions;

b) a second polypeptide comprising an antibody light chain construct, the antibody light chain construct comprising

i) a scFv, the scFv comprising

1) a first VH and a first VL,

wherein the first VH and the first VL associate to form a first antigen binding domain, and

2) a first linker peptide that connects the first VH and first VL; and

ii) an antibody light chain comprising a second VL and a CL;

wherein the scFv is attached at its C-terminus to the N-terminus of the second VL region of the antibody light chain;

c) a third polypeptide comprising an antibody heavy chain, the antibody heavy chain comprising a second VH, a second CH1 region, a second hinge region, a second CH2 region, and a second CH3 region,

wherein the second VH of the antibody heavy chain and the second VL of the antibody light chain associate to form a second antigen binding domain.

In one embodiment, the first linker comprises a sequence selected from the group consisting of: (Gly₃Ser)₂ (SEQ ID NO: 1), (Gly₄Ser)₂ (SEQ ID NO: 2), (Gly₃Ser)₃ (SEQ ID NO: 3), (Gly₄Ser)₃ (SEQ ID NO: 4), (Gly₃Ser)₄ (SEQ ID NO: 5), (Gly₄Ser)₄ (SEQ ID NO: 6), (Gly₃Ser)₅ (SEQ ID NO: 7), (Gly₄Ser)₅ (SEQ ID NO: 8), (Gly₃Ser)₆ (SEQ ID NO: 9), (Gly₄Ser)₆ (SEQ ID NO: 10), GSADDAKKDAAKKDAAKKDDAKKDDAGS (SEQ ID NO: 11), GSADDAKKDAAKKDAAKKDDAKKDDAKKDAGS (SEQ ID NO: 12), (Gly₃Gln)₂ (SEQ ID NO: 13), (Gly₄Gln)₂ (SEQ ID NO: 14), (Gly₃ Gln)₃ (SEQ ID NO: 15), (Gly₄Gln)₃ (SEQ ID NO: 16), (Gly₃Gln)₄ (SEQ ID NO: 17), (Gly₄Gln)₄ (SEQ ID NO: 18), (Gly₃Gln)₅ (SEQ ID NO: 19), (Gly₄Gln)₅ (SEQ ID NO: 20), (Gly₃Gln)₆ (SEQ ID NO: 21), and (Gly₄Gln)₆ (SEQ ID NO: 22).

In one embodiment, the scFv is attached to the antibody heavy chain via a second linker. In one embodiment, the second linker comprises a sequence selected from the group consisting of: (Gly₃Ser)₂ (SEQ ID NO: 1), (Gly₄Ser)₂ (SEQ ID NO: 2), (Gly₃Ser)₃ (SEQ ID NO: 3), (Gly₄Ser)₃ (SEQ ID NO: 4), (Gly₃Ser)₄ (SEQ ID NO: 5), (Gly₄Ser)₄ (SEQ ID NO: 6), (Gly₃Ser)₅ (SEQ ID NO: 7), (Gly₄Ser)₅ (SEQ ID NO: 8), (Gly₃Ser)₆ (SEQ ID NO: 9), and (Gly₄Ser)₆ (SEQ ID NO: 10),

In one embodiment, the scFv comprises the first VH attached at its C-terminus to the N-terminus of the first linker and the first linker is attached at its C-terminus to the N-terminus of the first VL. In another embodiment, the scFv comprises the first VL attached at its C-terminus to the N-terminus of the first linker and the first linker is attached at its C-terminus to the N-terminus of the first VH.

In one embodiment, the first antigen binding domain and the second antigen binding domain bind to epitopes on different polypeptides. In another embodiment, the first antigen binding domain and the second antigen binding domain bind to different epitopes on the same polypeptide. In one embodiment, the multispecific antibody construct is a biparatopic antibody construct.

In one embodiment, the Fc region consists of a hinge region, CH2 region, and CH3 regions.

In one embodiment, wherein the N-terminus of the Fc region is linked via its N-terminus to the C-terminus of the heavy chain via a third linker.

In one embodiment, the third linker comprises a sequence selected from the group consisting of: (Gly₃Ser)₂ (SEQ ID NO: 1), (Gly₄Ser)₂ (SEQ ID NO: 2), (Gly₃Ser)₃ (SEQ ID NO: 3), (Gly₄Ser)₃ (SEQ ID NO: 4), (Gly₃Ser)₄ (SEQ ID NO: 5), (Gly₄Ser)₄ (SEQ ID NO: 6), (Gly₃Ser)₅ (SEQ ID NO: 7), (Gly₄Ser)₅ (SEQ ID NO: 8), (Gly₃Ser)₆ (SEQ ID NO: 9), (Gly₄Ser)₆ (SEQ ID NO: 10), (Gly₃Gln)₂ (SEQ ID NO: 13), (Gly₄Gln)₂ (SEQ ID NO: 14), (Gly₃ Gln)₃ (SEQ ID NO: 15), (Gly₄Gln)₃ (SEQ ID NO: 16), (Gly₃Gln)₄ (SEQ ID NO: 17), (Gly₄Gln)₄ (SEQ ID NO: 18), (Gly₃Gln)₅ (SEQ ID NO: 19), (Gly₄Gln)₅ (SEQ ID NO: 20), (Gly₃Gln)₆ (SEQ ID NO: 21), and (Gly₄Gln)₆ (SEQ ID NO: 22).

In one embodiment, the first polypeptide consists of the antibody Fc region.

In one embodiment, the multispecific antibody construct is a bispecific antibody construct.

In one embodiment, the one CH3 domain comprises a F405L, F405A, F405D, F405E, F405H, F405I, F405K, F405M, F405N, F405Q, F4055, F405T, F405V, F405W, or F405Y mutation; and the other CH3 domain comprises a K409R mutation; wherein the numbering of amino acid residues is according to the EU index as set forth in Kabat.

In another embodiment, the one CH3 domain comprises a T366W mutation; and the other CH3 domain comprises T366S, L368A, Y407V mutations; wherein the numbering of amino acid residues is according to the EU index as set forth in Kabat.

In yet another embodiment, the one CH3 domain comprises K/R409D and K392D mutations; and the other CH3 domain comprises a D399K mutation; wherein the numbering of amino acid residues is according to the EU index as set forth in Kabat. In one embodiment, the CH3 domain that comprises a D399K mutation also comprises a E356K mutation; wherein the numbering of amino acid residues is according to the EU index as set forth in Kabat.

### BRIEF DESCRIPTION OF THE DRAWINGS

FIG. 1 depicts the crystal structure of 2 Fabs bound to 2 different domains in the same target protein. This provides the rational to design a novel format of multispecifics.

FIG. 2 depicts the schematic representation of a multispecific molecules comprised by three polypeptide chains. The scFv module can be connected via its C-ter VH to the N-ter of the Fab VH by flexible and/or rigid linkers.

FIG. 3 depicts the primary structure for the multispecific molecule showing the novel design implemented in chain A.

FIG. 4 depicts the schematic representation of multispecific molecules comprised by three polypeptide chains. The scFv module can be connected via its C-ter VH to the N-ter of the Fab VL by flexible and rigid linkers.

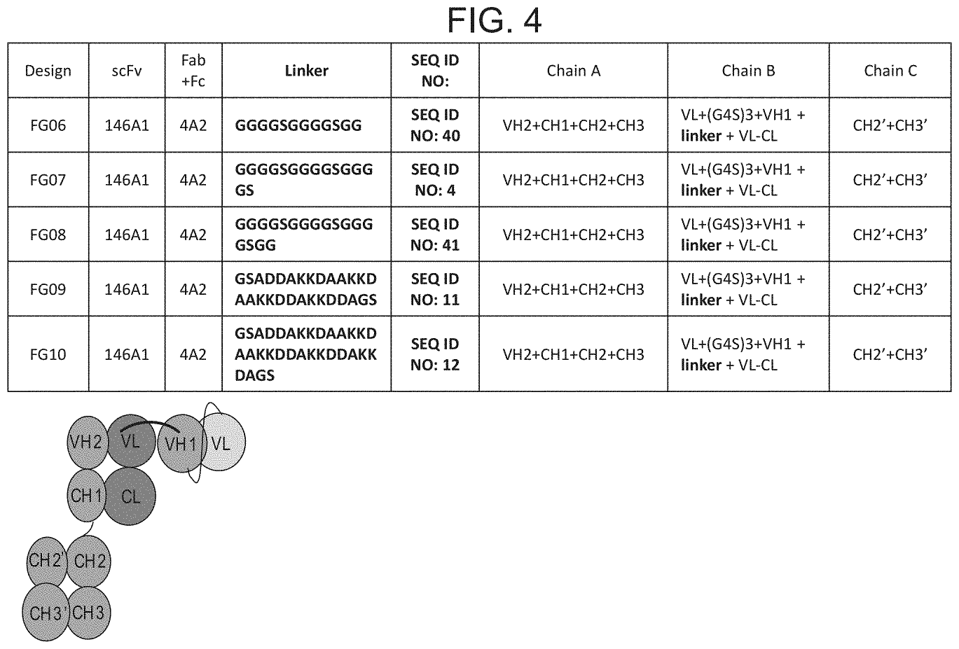

FIG. 5 depicts the schematic representation of multispecific molecules comprised by three polypeptide chains. The scFv module can be connected via its C-ter VH to the N-ter of the Fab VL by flexible and rigid linkers.

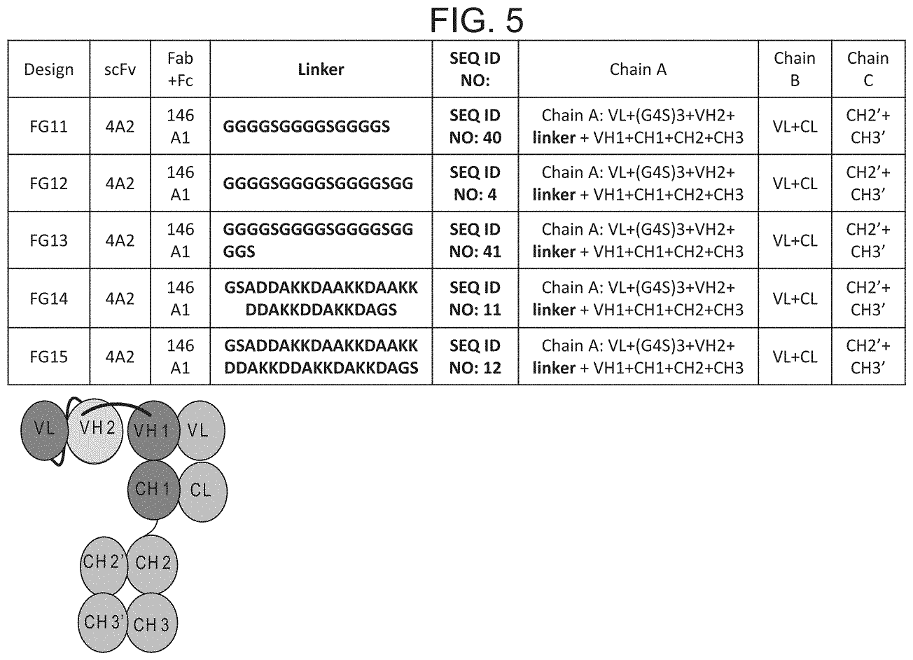

FIG. 6 depicts the expression and purification of Multispecific constructs.

FIG. 7 depicts the binding assay for the multispecific construct.

FIG. 8 depicts the cell binding assay for the Multispecific molecules.

### DETAILED DESCRIPTION

As used herein, the term “antigen binding protein” refers to a protein that specifically binds to one or more target antigens. An antigen binding protein can include an antibody and functional fragments thereof. A “functional antibody fragment” is a portion of an antibody that lacks at least some of the amino acids present in a full-length heavy chain and/or light chain, but which is still capable of specifically binding to an antigen. A functional antibody fragment includes, but is not limited to, a Fab fragment, a Fab′ fragment, a F(ab′)₂ fragment, a Fv fragment, a Fd fragment, and a complementarity determining region (CDR) fragment, and can be derived from any mammalian source, such as human, mouse, rat, rabbit, or camelid. Functional antibody fragments may compete for binding of a target antigen with an intact antibody and the fragments may be produced by the modification of intact antibodies (e.g. enzymatic or chemical cleavage) or synthesized de novo using recombinant DNA technologies or peptide synthesis.

“Heavy” and “light” chains refer to the two polypeptides which comprise an IgG. A heavy chain can be broken down into the following domains from N-terminus to C-terminus: VH, CH1, hinge, CH2, and CH3. A light chain can be broken down into the following domains from N-terminus to C-terminus: VL and CL. The CH1 and CL domains will interact such that the VH and VL domains form a functional conformation that binds to an antigen.

An antigen binding protein can also include a protein comprising one or more functional antibody fragments incorporated into a single polypeptide chain or into multiple polypeptide chains. A “multispecific antibody construct” is one or more polypeptides comprising one or more functional antibody portions that bind to two or more different antigens. A multispecific antibody construct can include a polypeptide that comprises a scFv connected to an antibody heavy or connected to an antibody light chain.

In certain aspects, the antigen binding proteins of the present invention are “multispecific” meaning that they are capable of specifically binding to two or more different antigens. In another aspect, the antigen binding proteins of the present invention are “bispecific” meaning that they are capable of specifically binding to two different antigens.

As used herein, an antigen binding protein “specifically binds” to a target antigen when it has a significantly higher binding affinity for, and consequently is capable of distinguishing, that antigen, compared to its affinity for other unrelated proteins, under similar binding assay conditions. Antigen binding proteins that specifically bind an antigen may have an equilibrium dissociation constant (K_{D})≤1×10⁻⁶ M. The antigen binding protein specifically binds antigen with “high affinity” when the K_{D} is ≤1×10⁻⁸ M. In one embodiment, the antigen binding proteins of the invention bind to target antigen(s) with a K_{D} of ≤5×10⁻⁷M. In another embodiment, the antigen binding proteins of the invention bind to target antigen(s) with a K_{D} of ≤1×10⁻⁷M.

Affinity is determined using a variety of techniques, an example of which is an affinity ELISA assay. In various embodiments, affinity is determined by a surface plasmon resonance assay (e.g., BIAcore®-based assay). Using this methodology, the association rate constant (kₐ in M⁻¹s⁻¹) and the dissociation rate constant (kₐ in s⁻¹) can be measured. The equilibrium dissociation constant (K_{D} in M) can then be calculated from the ratio of the kinetic rate constants (kₐ/kₐ). In some embodiments, affinity is determined by a kinetic method, such as a Kinetic Exclusion Assay (KinExA) as described in Rathanaswami et al. Analytical Biochemistry, Vol. 373:52-60, 2008. Using a KinExA assay, the equilibrium dissociation constant (K_{D} in M) and the association rate constant (kₐ in M⁻¹s⁻¹) can be measured. The dissociation rate constant (kₐ in s⁻¹) can be calculated from these values (K_{D}×kₐ). In other embodiments, affinity is determined by an equilibrium/solution method. In certain embodiments, affinity is determined by a FACS binding assay. In certain embodiments of the invention, the antigen binding protein specifically binds to target antigen(s) expressed by a mammalian cell (e.g., CHO, HEK 293, Jurkat), with a K_{D} of 20 nM (2.0×10⁻⁸ M) or less, K_{D} of 10 nM (1.0×10⁻⁸ M) or less, K_{D} of 1 nM (1.0×10⁻⁹ M) or less, K_{D} of 500 μM (5.0×10⁻¹⁰ M) or less, K_{D} of 200 μM (2.0×10⁻¹⁰ M) or less, K_{D} of 150 μM (1.50×10⁻¹⁰ M) or less, K_{D} of 125 μM (1.25×10⁻¹⁰M) or less, K_{D} of 105 μM (1.05×10⁻¹⁰ M) or less, K_{D} of 50 μM (5.0×10⁻¹¹ M) or less, or K_{D} of 20 μM (2.0×10⁻¹¹M) or less, as determined by a Kinetic Exclusion Assay, conducted by the method described in Rathanaswami et al. Analytical Biochemistry, Vol. 373:52-60, 2008. In some embodiments, the multispecific antibody constructs described herein exhibit desirable characteristics such as binding avidity as measured by kₐ (dissociation rate constant) for target antigen(s) of about 10⁻², 10⁻³, 10⁻⁴, 10⁻⁵, 10⁻⁶, 10⁻⁷, 10⁻⁸, 10⁻⁹, 10⁻¹⁰ s⁻¹ or lower (lower values indicating higher binding avidity), and/or binding affinity as measured by K_{D} (equilibrium dissociation constant) for target antigen(s) of about 10⁻⁹, 10⁻¹⁰, 10⁻¹¹, 10⁻¹², 10⁻¹³, 10⁻¹⁴, 10⁻¹⁵, 10⁻¹⁶ M or lower (lower values indicating higher binding affinity).

As used herein, the term “antigen binding domain,” which is used interchangeably with “binding domain,” refers to the region of the antigen binding protein that contains the amino acid residues that interact with the antigen and confer on the antigen binding protein its specificity and affinity for the antigen. As used herein, the term “target antigen(s)” refers, for example, to a first target antigen and/or a second target antigen of a bispecific molecule and also refers to a first target antigen, a second target antigen, a third target antigen, and/or a fourth target antigen of a tetraspecific molecule.

In certain embodiments of the multispecific antibody constructs of the present invention, the binding domain may be derived from an antibody or functional fragment thereof. For instance, the binding domains of the multispecific antibody constructs of the invention may comprise one or more complementarity determining regions (CDR) derived from the light and heavy chain variable regions of antibodies that specifically bind to target antigen(s). As used herein, the term “CDR” refers to the complementarity determining region (also termed “minimal recognition units” or “hypervariable region”) within antibody variable sequences. There are three heavy chain variable region CDRs (CDRH1, CDRH2 and CDRH3) and three light chain variable region CDRs (CDRL1, CDRL2 and CDRL3). The term “CDR region” as used herein refers to a group of three CDRs that occur in a single variable region (i.e. the three light chain CDRs or the three heavy chain CDRs). The CDRs in each of the two chains typically are aligned by the framework regions to form a structure that binds specifically with a specific epitope or domain on the target protein. From N-terminus to C-terminus, naturally-occurring light and heavy chain variable regions both typically conform with the following order of these elements: FR1, CDR1, FR2, CDR2, FR3, CDR3 and FR4.

Both the EU index as in Kabat et al., Sequences of Proteins of Immunological Interest, 5th Ed. Public Health Service, National Institutes of Health, Bethesda, Md. (1991) and AHo numbering schemes (Honegger A. and Plûckthun A. J Mol Biol. 2001 Jun. 8; 309(3):657-70) can be used in the present invention. Amino acid positions and complementarity determining regions (CDRs) and framework regions (FR) of a given antibody may be identified using either system. For example, EU heavy chain positions of 39, 44, 183, 356, 357, 370, 392, 399, and 409 are equivalent to AHo heavy chain positions 46, 51, 230, 484, 485, 501, 528, 535, and 551, respectively. Similarly, EU light chain positions 38, 100, and 176 are equivalent to AHO light chain positions 46 141, and 230, respectively.

Papain digestion of antibodies produces two identical antigen-binding fragments, called “Fab” fragments, each with a single antigen-binding site, and a residual “Fc” fragment which contains the immunoglobulin constant region. The Fab fragment contains all of the variable domain, as well as the constant domain of the light chain and the first constant domain (CH1) of the heavy chain. Thus, a “Fab fragment” is comprised of one immunoglobulin light chain (light chain variable region (VL) and constant region (CL)) and the CH1 region and variable region (VH) of one immunoglobulin heavy chain. The heavy chain of a Fab molecule cannot form a disulfide bond with another heavy chain molecule. The Fc fragment displays carbohydrates and is responsible for many antibody effector functions (such as binding complement and cell receptors), that distinguish one class of antibody from another. The “Fd fragment” comprises the VH and CH1 domains from an immunoglobulin heavy chain. The Fd fragment represents the heavy chain component of the Fab fragment.

A “Fab′ fragment” is a Fab fragment having at the C-terminus of the CH1 domain one or more cysteine residues from the antibody hinge region.

A “F(ab)₂ fragment” is a bivalent fragment including two Fab′ fragments linked by a disulfide bridge between the heavy chains at the hinge region.

The “Fv” fragment is the minimum fragment that contains a complete antigen recognition and binding site from an antibody. This fragment consists of a dimer of one immunoglobulin heavy chain variable region (VH) and one immunoglobulin light chain variable region (VL) in tight, non-covalent association. It is in this configuration that the three CDRs of each variable region interact to define an antigen binding site on the surface of the VH-VL dimer. A single light chain or heavy chain variable region (or half of an Fv fragment comprising only three CDRs specific for an antigen) has the ability to recognize and bind antigen, although at a lower affinity than the entire binding site comprising both VH and VL.

The “variable region,” used interchangeably herein with “variable domain” (variable region of a light chain (VL), variable region of a heavy chain (VH)) refers to the region in each of the light and heavy immunoglobulin chains which is involved directly in binding the antibody to the antigen. As discussed above, the regions of variable light and heavy chains have the same general structure and each region comprises four framework (FR) regions whose sequences are widely conserved, connected by three CDRs. The framework regions adopt a beta-sheet conformation and the CDRs may form loops connecting the beta-sheet structure. The CDRs in each chain are held in their three-dimensional structure by the framework regions and form, together with the CDRs from the other chain, the antigen binding site.

The “immunoglobulin domain” represents a peptide comprising an amino acid sequence similar to that of immunoglobulin and comprising approximately 100 amino acid residues including at least two cysteine residues. Examples of the immunoglobulin domain include VH, CH1, CH2, and CH3 of an immunoglobulin heavy chain, and VL and CL of an immunoglobulin light chain. In addition, the immunoglobulin domain is found in proteins other than immunoglobulin. Examples of the immunoglobulin domain in proteins other than immunoglobulin include an immunoglobulin domain included in a protein belonging to an immunoglobulin super family, such as a major histocompatibility complex (MHC), CD1, B7, T-cell receptor (TCR), and the like. Any of the immunoglobulin domains can be used as an immunoglobulin domain for the multivalent antibody of the present invention.

In a human antibody, CH1 means a region having the amino acid sequence at positions 118 to 215 of the EU index. A highly flexible amino acid region called a “hinge region” exists between CH1 and CH2. CH2 represents a region having the amino acid sequence at positions 231 to 340 of the EU index, and CH3 represents a region having the amino acid sequence at positions 341 to 446 of the EU index.

“CL” represents a constant region of a light chain. In the case of a κ chain of a human antibody, CL represents a region having the amino acid sequence at positions 108 to 214 of the EU index. In a λ chain, CL represents a region having the amino acid sequence at positions 108 to 215.

The binding domains that specifically bind to target antigen(s) can be derived a) from known antibodies to these antigens or b) from new antibodies or antibody fragments obtained by de novo immunization methods using the antigen proteins or fragments thereof, by phage display, or other routine methods. The antibodies from which the binding domains for the multispecific antibody constructs are derived can be monoclonal antibodies, polyclonal antibodies, recombinant antibodies, human antibodies, or humanized antibodies. In certain embodiments, the antibodies from which the binding domains are derived are monoclonal antibodies. In these and other embodiments, the antibodies are human antibodies or humanized antibodies and can be of the IgG1-, IgG2-, IgG3-, or IgG4-type.

The term “monoclonal antibody” (or “mAb”) as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against an individual antigenic site or epitope, in contrast to polyclonal antibody preparations that typically include different antibodies directed against different epitopes. Monoclonal antibodies may be produced using any technique known in the art, e.g., by immortalizing spleen cells harvested from the transgenic animal after completion of the immunization schedule. The spleen cells can be immortalized using any technique known in the art, e.g., by fusing them with myeloma cells to produce hybridomas. Myeloma cells for use in hybridoma-producing fusion procedures are non-antibody-producing, have high fusion efficiency, and enzyme deficiencies that render them incapable of growing in certain selective media which support the growth of only the desired fused cells (hybridomas). Examples of suitable cell lines for use in mouse fusions include Sp-20, P3-X63/Ag8, P3-X63-Ag8.653, NS1/1.Ag 4 1, Sp210-Ag14, FO, NSO/U, MPC-11, MPC11-X45-GTG 1.7 and S194/5XXO Bul; examples of cell lines used in rat fusions include R210.RCY3, Y3-Ag 1.2.3, IR983F and 4B210. Other cell lines useful for cell fusions are U-266, GM1500-GRG2, LICR-LON-HMy2 and UC729-6.

In some instances, a hybridoma cell line is produced by immunizing an animal (e.g., a transgenic animal having human immunoglobulin sequences) with a target antigen(s) immunogen; harvesting spleen cells from the immunized animal; fusing the harvested spleen cells to a myeloma cell line, thereby generating hybridoma cells; establishing hybridoma cell lines from the hybridoma cells, and identifying a hybridoma cell line that produces an antibody that binds target antigen(s).

Monoclonal antibodies secreted by a hybridoma cell line can be purified using any technique known in the art, such as protein A-Sepharose, hydroxylapatite chromatography, gel electrophoresis, dialysis, or affinity chromatography. Hybridomas or mAbs may be further screened to identify mAbs with particular properties, such as the ability to bind cells expressing target antigen(s), ability to block or interfere with the binding of target antigen(s) to their respective receptors or ligands, or the ability to functionally block either of target antigen(s).

In some embodiments, the binding domains of the multispecific antibody constructs of the invention may be derived from humanized antibodies against target antigen(s). A “humanized antibody” refers to an antibody in which regions (e.g. framework regions) have been modified to comprise corresponding regions from a human immunoglobulin. Generally, a humanized antibody can be produced from a monoclonal antibody raised initially in a non-human animal. Certain amino acid residues in this monoclonal antibody, typically from non-antigen recognizing portions of the antibody, are modified to be homologous to corresponding residues in a human antibody of corresponding isotype. Humanization can be performed, for example, using various methods by substituting at least a portion of a rodent variable region for the corresponding regions of a human antibody (see, e.g., U.S. Pat. Nos. 5,585,089 and 5,693,762; Jones et al., Nature, Vol. 321:522-525, 1986; Riechmann et al., Nature, Vol. 332:323-27, 1988; Verhoeyen et al., Science, Vol. 239:1534-1536, 1988). The CDRs of light and heavy chain variable regions of antibodies generated in another species can be grafted to consensus human FRs. To create consensus human FRs, FRs from several human heavy chain or light chain amino acid sequences may be aligned to identify a consensus amino acid sequence.

New antibodies generated against the target antigen(s) from which binding domains for the multispecific antibody constructs of the invention can be derived can be fully human antibodies. A “fully human antibody” is an antibody that comprises variable and constant regions derived from human germ line immunoglobulin sequences. One specific means provided for implementing the production of fully human antibodies is the “humanization” of the mouse humoral immune system. Introduction of human immunoglobulin (Ig) loci into mice in which the endogenous Ig genes have been inactivated is one means of producing fully human monoclonal antibodies (mAbs) in mouse, an animal that can be immunized with any desirable antigen. Using fully human antibodies can minimize the immunogenic and allergic responses that can sometimes be caused by administering mouse or mouse-derived mAbs to humans as therapeutic agents.

Fully human antibodies can be produced by immunizing transgenic animals (usually mice) that are capable of producing a repertoire of human antibodies in the absence of endogenous immunoglobulin production. Antigens for this purpose typically have six or more contiguous amino acids, and optionally are conjugated to a carrier, such as a hapten. See, e.g., Jakobovits et al., 1993, Proc. Natl. Acad. Sci. USA 90:2551-2555; Jakobovits et al., 1993, Nature 362:255-258; and Bruggermann et al., 1993, Year in Immunol. 7:33. In one example of such a method, transgenic animals are produced by incapacitating the endogenous mouse immunoglobulin loci encoding the mouse heavy and light immunoglobulin chains therein, and inserting into the mouse genome large fragments of human genome DNA containing loci that encode human heavy and light chain proteins. Partially modified animals, which have less than the full complement of human immunoglobulin loci, are then cross-bred to obtain an animal having all of the desired immune system modifications. When administered an immunogen, these transgenic animals produce antibodies that are immunospecific for the immunogen but have human rather than murine amino acid sequences, including the variable regions. For further details of such methods, see, for example, WO96/33735 and WO94/02602. Additional methods relating to transgenic mice for making human antibodies are described in U.S. Pat. Nos. 5,545,807; 6,713,610; 6,673,986; 6,162,963; 5,939,598; 5,545,807; 6,300,129; 6,255,458; 5,877,397; 5,874,299 and 5,545,806; in PCT publications WO91/10741, WO90/04036, WO 94/02602, WO 96/30498, WO 98/24893 and in EP 546073B1 and EP 546073A1.

The transgenic mice described above, referred to herein as “HuMab” mice, contain a human immunoglobulin gene minilocus that encodes unrearranged human heavy (mu and gamma) and kappa light chain immunoglobulin sequences, together with targeted mutations that inactivate the endogenous mu and kappa chain loci (Lonberg et al., 1994, Nature 368:856-859). Accordingly, the mice exhibit reduced expression of mouse IgM or kappa and in response to immunization, and the introduced human heavy and light chain transgenes undergo class switching and somatic mutation to generate high affinity human IgG kappa monoclonal antibodies (Lonberg et al., supra.; Lonberg and Huszar, 1995, Intern. Rev. Immunol. 13: 65-93; Harding and Lonberg, 1995, Ann. N.Y Acad. Sci. 764:536-546). The preparation of HuMab mice is described in detail in Taylor et al., 1992, Nucleic Acids Research 20:6287-6295; Chen et al., 1993, International Immunology 5:647-656; Tuaillon et al., 1994, J. Immunol. 152:2912-2920; Lonberg et al., 1994, Nature 368:856-859; Lonberg, 1994, Handbook of Exp. Pharmacology 113:49-101; Taylor et al., 1994, International Immunology 6:579-591; Lonberg and Huszar, 1995, Intern. Rev. Immunol. 13:65-93; Harding and Lonberg, 1995, Ann. N.Y Acad. Sci. 764:536-546; Fishwild et al., 1996, Nature Biotechnology 14:845-851; the foregoing references are hereby incorporated by reference in their entirety for all purposes. See, further U.S. Pat. Nos. 5,545,806; 5,569,825; 5,625,126; 5,633,425; 5,789,650; 5,877,397; 5,661,016; 5,814,318; 5,874,299; and 5,770,429; as well as U.S. Pat. No. 5,545,807; International Publication Nos. WO 93/1227; WO 92/22646; and WO 92/03918, the disclosures of all of which are hereby incorporated by reference in their entirety for all purposes. Technologies utilized for producing human antibodies in these transgenic mice are disclosed also in WO 98/24893, and Mendez et al., 1997, Nature Genetics 15:146-156, which are hereby incorporated by reference.

Human-derived antibodies can also be generated using phage display techniques. Phage display is described in e.g., Dower et al., WO 91/17271, McCafferty et al., WO 92/01047, and Caton and Koprowski, Proc. Natl. Acad. Sci. USA, 87:6450-6454 (1990), each of which is incorporated herein by reference in its entirety. The antibodies produced by phage technology are usually produced as antigen binding fragments, e.g. Fv or Fab fragments, in bacteria and thus lack effector functions. Effector functions can be introduced by one of two strategies: The fragments can be engineered either into complete antibodies for expression in mammalian cells, or into multispecific antibody fragments with a second binding site capable of triggering an effector function, if desired. Typically, the Fd fragment (VH-CH1) and light chain (VL-CL) of antibodies are separately cloned by PCR and recombined randomly in combinatorial phage display libraries, which can then be selected for binding to a particular antigen. The antibody fragments are expressed on the phage surface, and selection of Fv or Fab (and therefore the phage containing the DNA encoding the antibody fragment) by antigen binding is accomplished through several rounds of antigen binding and re-amplification, a procedure termed panning. Antibody fragments specific for the antigen are enriched and finally isolated. Phage display techniques can also be used in an approach for the humanization of rodent monoclonal antibodies, called “guided selection” (see Jespers, L. S., et al., Bio/Technology 12, 899-903 (1994)). For this, the Fd fragment of the mouse monoclonal antibody can be displayed in combination with a human light chain library, and the resulting hybrid Fab library may then be selected with antigen. The mouse Fd fragment thereby provides a template to guide the selection. Subsequently, the selected human light chains are combined with a human Fd fragment library. Selection of the resulting library yields entirely human Fab.

The term “identity,” as used herein, refers to a relationship between the sequences of two or more polypeptide molecules or two or more nucleic acid molecules, as determined by aligning and comparing the sequences. “Percent identity,” as used herein, means the percent of identical residues between the amino acids or nucleotides in the compared molecules and is calculated based on the size of the smallest of the molecules being compared. For these calculations, gaps in alignments (if any) must be addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073. For example, sequence identity can be determined by standard methods that are commonly used to compare the similarity in position of the amino acids of two polypeptides. Using a computer program such as BLAST or FASTA, two polypeptide or two polynucleotide sequences are aligned for optimal matching of their respective residues (either along the full length of one or both sequences, or along a pre-determined portion of one or both sequences). The programs provide a default opening penalty and a default gap penalty, and a scoring matrix such as PAM 250 (a standard scoring matrix; see Dayhoff et al., in Atlas of Protein Sequence and Structure, vol. 5, supp. 3 (1978)) can be used in conjunction with the computer program. For example, the percent identity can then be calculated as: the total number of identical matches multiplied by 100 and then divided by the sum of the length of the longer sequence within the matched span and the number of gaps introduced into the longer sequences in order to align the two sequences. In calculating percent identity, the sequences being compared are aligned in a way that gives the largest match between the sequences.

The GCG program package is a computer program that can be used to determine percent identity, which package includes GAP (Devereux et al., 1984, Nucl. Acid Res. 12:387; Genetics Computer Group, University of Wisconsin, Madison, Wis.). The computer algorithm GAP is used to align the two polypeptides or two polynucleotides for which the percent sequence identity is to be determined. The sequences are aligned for optimal matching of their respective amino acid or nucleotide (the “matched span”, as determined by the algorithm). A gap opening penalty (which is calculated as 3× the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm.

Recommended parameters for determining percent identity for polypeptides or nucleotide sequences using the GAP program include the following:

Algorithm: Needleman et al., 1970, J. Mol. Biol. 48:443-453;

Comparison matrix: BLOSUM 62 from Henikoff et al., 1992, supra;

Gap Penalty: 12 (but with no penalty for end gaps)

Gap Length Penalty: 4

Threshold of Similarity: 0

Certain alignment schemes for aligning two amino acid sequences may result in matching of only a short region of the two sequences, and this small aligned region may have very high sequence identity even though there is no significant relationship between the two full-length sequences. Accordingly, the selected alignment method (GAP program) can be adjusted if so desired to result in an alignment that spans at least 50 contiguous amino acids of the target polypeptide.

As used herein, the term “antibody” refers to a tetrameric immunoglobulin protein comprising two light chain polypeptides (about 25 kDa each) and two heavy chain polypeptides (about 50-70 kDa each). The term “light chain” or “immunoglobulin light chain” refers to a polypeptide comprising, from amino terminus to carboxyl terminus, a single immunoglobulin light chain variable region (VL) and a single immunoglobulin light chain constant domain (CL). The immunoglobulin light chain constant domain (CL) can be kappa (κ) or lambda (λ). The term “heavy chain” or “immunoglobulin heavy chain” refers to a polypeptide comprising, from amino terminus to carboxyl terminus, a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4). Heavy chains are classified as mu (μ), delta (Δ), gamma (γ), alpha (α), and epsilon (ε), and define the antibody's isotype as IgM, IgD, IgG, IgA, and IgE, respectively. The IgG-class and IgA-class antibodies are further divided into subclasses, namely, IgG1, IgG2, IgG3, and IgG4, and IgA1 and IgA2, respectively. The heavy chains in IgG, IgA, and IgD antibodies have three domains (CH1, CH2, and CH3), whereas the heavy chains in IgM and IgE antibodies have four domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (i.e. between the light and heavy chain) and between the hinge regions of the antibody heavy chains.

The multispecific antibody constructs can comprise any immunoglobulin constant region. The term “constant region” as used herein refers to all domains of an antibody other than the variable region. The constant region is not involved directly in binding of an antigen, but exhibits various effector functions. As described above, antibodies are divided into particular isotypes (IgA, IgD, IgE, IgG, and IgM) and subtypes (IgG1, IgG2, IgG3, IgG4, IgA1 IgA2) depending on the amino acid sequence of the constant region of their heavy chains. The light chain constant region can be, for example, a kappa- or lambda-type light chain constant region, e.g., a human kappa- or lambda-type light chain constant region, which are found in all five antibody isotypes. Examples of human immunoglobulin light chain constant region sequences are shown in the following table.

TABLE 4
Exemplary Human Immunoglobulin Light Chain Constant Regions

| | SEQ | |
|---|---|---|
| | ID | |
| Designation | NO: | CL Domain Amino Acid Sequence |
| CL-1 | 23 | GQPKANPTVTLFPPSSEELQANKATLVCLISDFYPGAVTVAWKA |
| | | DGSPVKAGVETTKPSKQSNNKYAASSYLSLTPEQWKSHRSYSC |
| | | QVTHEGSTVEKTVAPTECS |
| CL-2 | 24 | GQPKAAPSVTLFPPSSEELQANKATLVCLISDFYPGAVTVAWKA |
| | | DSSPVKAGVETTTPSKQSNNKYAASSYLSLTPEQWKSHRSYSCQ |
| | | VTHEGSTVEKTVAPTECS |
| CL-3 | 25 | GQPKAAPSVTLFPPSSEELQANKATLVCLISDFYPGAVTVAWKA |
| | | DSSPVKAGVETTTPSKQSNNKYAASSYLSLTPEQWKSHKSYSCQ |
| | | VTHEGSTVEKTVAPTECS |
| CL-7 | 26 | GQPKAAPSVTLFPPSSEELQANKATLVCLVSDFYPGAVTVAWK |
| | | ADGSPVKVGVETTKPSKQSNNKYAASSYLSLTPEQWKSHRSYS |
| | | CRVTHEGSTVEKTVAPAECS |

The heavy chain constant region of the multispecific antibody constructs can be, for example, an alpha-, delta-, epsilon-, gamma-, or mu-type heavy chain constant region, e.g., a human alpha-, delta-, epsilon-, gamma-, or mu-type heavy chain constant region. In some embodiments, the multispecific antibody constructs comprise a heavy chain constant region from an IgG1, IgG2, IgG3, or IgG4 immunoglobulin. In one embodiment, the multispecific antibody construct comprises a heavy chain constant region from a human IgG1 immunoglobulin. In another embodiment, the multispecific antibody construct comprises a heavy chain constant region from a human IgG2 immunoglobulin. Examples of human IgG1 and IgG2 heavy chain constant region sequences are shown below in Table 5.

TABLE 5
Exemplary Human Immunoglobulin Heavy Chain Constant Regions

| | SEQ | |
|---|---|---|
| | ID | |
| Ig isotype | NO: | Heavy Chain Constant Region Amino Acid Sequence |
| Human | 27 | ASTKGPSVFPLAPSSKSTSGGTAALGCLVKDYFPEPVTVSWNSGALTSGVHTFPAV |
| IgG1z | | LQSSGLYSLSSVVTVPSSSLGTQTYICNVNHKPSNTKVDKKVEPKSCDKTHTCPPCP |
| | | APELLGGPSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEKTISKAK |
| | | GQPREPQVYTLPPSREEMTKNQVSLTCLVKGFYPSDIAVEWESNGQPENNYKTTPP |
| | | VLDSDGSFFLYSKLTVDKSRWQQGNVFSCSVMHEALHNHYTQKSLSLSPGK |
| Human | 28 | ASTKGPSVFPLAPSSKSTSGGTAALGCLVKDYFPEPVTVSWNSGALTSGVHTFPAV |
| IgG1za | | LQSSGLYSLSSVVTVPSSSLGTQTYICNVNHKPSNTKVDKKVEPKSCDKTHTCPPCP |
| | | APELLGGPSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEKTISKAK |
| | | GQPREPQVYTLPPSRDELTKNQVSLTCLVKGFYPSDIAVEWESNGQPENNYKTTPP |
| | | VLDSDGSFFLYSKLTVDKSRWQQGNVFSCSVMHEALHNHYTQKSLSLSPGK |
| Human | 29 | ASTKGPSVFPLAPSSKSTSGGTAALGCLVKDYFPEPVTVSWNSGALTSGVHTFPAV |
| IgG1f | | LQSSGLYSLSSVVTVPSSSLGTQTYICNVNHKPSNTKVDKRVEPKSCDKTHTCPPCP |
| | | APELLGGPSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEKTISKAK |
| | | GQPREPQVYTLPPSREEMTKNQVSLTCLVKGFYPSDIAVEWESNGQPENNYKTTPP |
| | | VLDSDGSFFLYSKLTVDKSRWQQGNVFSCSVMHEALHNHYTQKSLSLSPGK |
| Human | 30 | ASTKGPSVFPLAPSSKSTSGGTAALGCLVKDYFPEPVTVSWNSGALTSGVHTFPAV |
| IgG1fa | | LQSSGLYSLSSVVTVPSSSLGTQTYICNVNHKPSNTKVDKRVEPKSCDKTHTCPPCP |
| | | APELLGGPSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEKTISKAK |
| | | GQPREPQVYTLPPSRDELTKNQVSLTCLVKGFYPSDIAVEWESNGQPENNYKTTPP |
| | | VLDSDGSFFLYSKLTVDKSRWQQGNVFSCSVMHEALHNHYTQKSLSLSPGK |
| Human | 31 | ASTKGPSVFPLAPCSRSTSESTAALGCLVKDYFPEPVTVSWNSGALTSGVHTFPAV |
| IgG2 | | LQSSGLYSLSSVVTVPSSNFGTQTYTCNVDHKPSNTKVDKTVERKCCVECPPCPAP |
| | | PVAGPSVFLFPPKPKDTLMISRTPEVTCVVVDVSHEDPEVQFNWYVDGVEVHNAK |
| | | TKPREEQFNSTFRVVSVLTVVHQDWLNGKEYKCKVSNKGLPAPIEKTISKTKGQPR |
| | | EPQVYTLPPSREEMTKNQVSLTCLVKGFYPSDIAVEWESNGQPENNYKTTPPMLDS |
| | | DGSFFLYSKLTVDKSRWQQGNVFSCSVMHEALHNHYTQKSLSLSPGK |

A variable region may be attached to the above light and heavy chain constant regions to form complete antibody light and heavy chains, respectively. Further, each of the so generated heavy and light chain polypeptides may be combined to form a multispecific antibody construct. It should be understood that the heavy chain and light chain variable regions provided herein can also be attached to other constant domains having different sequences than the exemplary sequences listed above.

In certain embodiments of the invention two different Fc domains are used to form the a heterodimeric molecule of the present invention. To facilitate assembly of the Fc and heavy chains into a multispecific antibody construct, the Fc domain and the Fc domain of the heavy chain from each the Fc-containing polypeptides of the multispecific antibody construct can be engineered to reduce the formation of mispaired molecules. For example, one approach to promote heterodimer Fc formation over homodimer Fc formation is the so-called “knobs-into-holes” method, which involves introducing mutations into the CH3 domains of two different antibody heavy chain Fc regions at the contact interface. Specifically, one or more bulky amino acids in one antibody heavy chain Fc region is replaced with amino acids having short side chains (e.g. alanine or threonine) to create a “hole,” whereas one or more amino acids with large side chains (e.g. tyrosine or tryptophan) are introduced into the other heavy chain to create a “knob.” When the modified heavy chain Fc regions are co-expressed, a greater percentage of heterodimers (knob-hole) are formed as compared to homodimers (hole-hole or knob-knob). The “knobs-into-holes” methodology is described in detail in WO 96/027011; Ridgway et al., Protein Eng., Vol. 9: 617-621, 1996; and Merchant et al., Nat, Biotechnol., Vol. 16: 677-681, 1998, all of which are hereby incorporated by reference in their entireties.

Another approach for promoting heterodimer formation to the exclusion of homodimer formation entails utilizing an electrostatic steering mechanism (see Gunasekaran et al., J. Biol. Chem., Vol. 285: 19637-19646, 2010, which is hereby incorporated by reference in its entirety). This approach involves introducing or exploiting charged residues in the CH3 domain in each Fc region so that the two different Fc regions associate through opposite charges that cause electrostatic attraction. Homodimerization of the identical Fc regions are disfavored because the identical Fc regions have the same charge and therefore are repelled. The electrostatic steering technique and suitable charge pair mutations for promoting heterodimers and correct light chain/heavy chain pairing is described in WO2009089004 and WO2014081955, both of which are hereby incorporated by reference in their entireties.

In certain embodiments, amino acids (e.g. lysine) at one or more positions of one CH3 domain are selected from 370, 392 and 409 (EU numbering system) are replaced with a negatively-charged amino acid (e.g., aspartic acid and glutamic acid) and amino acids (e.g., aspartic acid or glutamic acid) at one or more positions selected from 356, 357, and 399 (EU numbering system) of the other CH3 domain are replaced with a positively-charged amino acid (e.g., lysine, histidine and arginine).

In particular embodiments, the multispecific antibody construct comprises a first CH3-containing polypeptide (a heavy chain or an Fc domain) comprising negatively-charged amino acids at positions 392 and 409 (e.g., K392D and K409D substitutions), and a second CH3-containing polypeptide comprising positively-charged amino acids at positions 356 and 399 (e.g., E356K and D399K substitutions). In other particular embodiments, the multispecific antibody construct comprises a first CH3-containing polypeptide comprising negatively-charged amino acids at positions 392, 409, and 370 (e.g., K392D, K409D, and K370D substitutions), and a second CH3-containing polypeptide comprising positively-charged amino acids at positions 356, 399, and 357 (e.g., E356K, D399K, and E357K substitutions).

In one embodiment, the problem of mispairing is avoided by connecting the Fc domain to the heavy chain via a linker. In such instances the heavy chain and the Fc domain can form a single chain Fc (scFc).

Any of the constant domains can be modified to contain one or more of the charge pair mutations described above to facilitate correct assembly of a multispecific antibody construct.

The inventive multispecific antibody constructs also encompass antibodies comprising the heavy chain(s) and/or light chain(s), where one, two, three, four or five amino acid residues are lacking from the N-terminus or C-terminus, or both, in relation to any one of the heavy and light chains, e.g., due to post-translational modifications resulting from the type of host cell in which the antibodies are expressed. For instance, Chinese Hamster Ovary (CHO) cells frequently cleave off a C-terminal lysine from antibody heavy chains.

As used herein, the term “Fc region” refers to the C-terminal region of an immunoglobulin heavy chain which may be generated by papain digestion of an intact antibody. The Fc region of an immunoglobulin generally comprises two constant domains, a CH2 domain and a CH3 domain, and optionally comprises a hinge domain. In certain embodiments, the Fc region is an Fc region from an IgG1, IgG2, IgG3, or IgG4 immunoglobulin. In some embodiments, the Fc region comprises CH2 and CH3 domains from a human IgG1 or human IgG2 immunoglobulin. The Fc region may retain effector function, such as C1q binding, complement dependent cytotoxicity (CDC), Fc receptor binding, antibody-dependent cell-mediated cytotoxicity (ADCC), and phagocytosis. In other embodiments, the Fc region may be modified to reduce or eliminate effector function as described in further detail herein.

In certain embodiments of the multispecific antibody constructs of the invention, the binding domain positioned at the amino terminus of the Fc region (i.e. the amino-terminal binding domain) is a Fab fragment fused to the amino terminus of the Fc region through a peptide linker described herein or through an immunoglobulin hinge region. An “immunoglobulin hinge region” refers to the amino acid sequence connecting the CH1 domain and the CH2 domain of an immunoglobulin heavy chain. The hinge region of human IgG1 is generally defined as the amino acid sequence from about Glu216 or about Cys226, to about Pro230. Hinge regions of other IgG isotypes may be aligned with the IgG1 sequence by placing the first and last cysteine residues forming inter-heavy chain disulfide bonds in the same positions and are determinable to those of skill in the art. In some embodiments, the amino-terminal binding domain is joined to the amino terminus of the Fc region through a human IgG1 hinge region. In other embodiments, the amino-terminal binding domain is joined to the amino terminus of the Fc region through a human IgG2 hinge region. In one embodiment, the amino-terminal binding domain (e.g. Fab fragment) is fused to the Fc region through the carboxyl terminus of the CH1 region of the Fab.

As used herein, the term “modified heavy chain” refers to a fusion protein comprising an immunoglobulin heavy chain, particularly a human IgG1 or human IgG2 heavy chain, and a functional antibody fragment (e.g. scFv), wherein the fragment or portion thereof is fused, optionally through a peptide linker, to the N-terminus or C-terminus of the heavy chain.

As used herein, the term “modified light chain” refers to a fusion protein comprising an immunoglobulin light chain and a functional antibody fragment (e.g. scFv), wherein the fragment or portion thereof is fused, optionally through a peptide linker, to the N-terminus or C-terminus of the light chain.

The heavy chain constant regions or the Fc regions of the multispecific antibody constructs described herein may comprise one or more amino acid substitutions that affect the glycosylation and/or effector function of the antigen binding protein. One of the functions of the Fc region of an immunoglobulin is to communicate to the immune system when the immunoglobulin binds its target. This is commonly referred to as “effector function.” Communication leads to antibody-dependent cellular cytotoxicity (ADCC), antibody-dependent cellular phagocytosis (ADCP), and/or complement dependent cytotoxicity (CDC). ADCC and ADCP are mediated through the binding of the Fc region to Fc receptors on the surface of cells of the immune system. CDC is mediated through the binding of the Fc with proteins of the complement system, e.g., C1q. In some embodiments, the multispecific antibody constructs of the invention comprise one or more amino acid substitutions in the constant region to enhance effector function, including ADCC activity, CDC activity, ADCP activity, and/or the clearance or half-life of the antigen binding protein. Exemplary amino acid substitutions (EU numbering) that can enhance effector function include, but are not limited to, E233L, L234I, L234Y, L235S, G236A, S239D, F243L, F243V, P247I, D280H, K290S, K290E, K290N, K290Y, R292P, E294L, Y296W, S298A, S298D, S298V, S298G, S298T, T299A, Y300L, V3051, Q311M, K326A, K326E, K326W, A330S, A330L, A330M, A330F, 1332E, D333A, E333S, E333A, K334A, K334V, A339D, A339Q, P396L, or combinations of any of the foregoing.

In other embodiments, the multispecific antibody constructs of the invention comprise one or more amino acid substitutions in the constant region to reduce effector function. Exemplary amino acid substitutions (EU numbering) that can reduce effector function include, but are not limited to, C220S, C226S, C229S, E233P, L234A, L234V, V234A, L234F, L235A, L235E, G237A, P238S, S267E, H268Q, N297A, N297G, V309L, E318A, L328F, A330S, A331S, P331S or combinations of any of the foregoing.

Glycosylation can contribute to the effector function of antibodies, particularly IgG1 antibodies. Thus, in some embodiments, the multispecific antibody constructs of the invention may comprise one or more amino acid substitutions that affect the level or type of glycosylation of the binding proteins. Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

In certain embodiments, glycosylation of the multispecific antibody constructs described herein is increased by adding one or more glycosylation sites, e.g., to the Fc region of the binding protein. Addition of glycosylation sites to the antigen binding protein can be conveniently accomplished by altering the amino acid sequence such that it contains one or more of the above-described tri-peptide sequences (for N-linked glycosylation sites). The alteration may also be made by the addition of, or substitution by, one or more serine or threonine residues to the starting sequence (for O-linked glycosylation sites). For ease, the antigen binding protein amino acid sequence may be altered through changes at the DNA level, particularly by mutating the DNA encoding the target polypeptide at preselected bases such that codons are generated that will translate into the desired amino acids.

The invention also encompasses production of bispecific antigen binding protein molecules with altered carbohydrate structure resulting in altered effector activity, including antigen binding proteins with absent or reduced fucosylation that exhibit improved ADCC activity. Various methods are known in the art to reduce or eliminate fucosylation. For example, ADCC effector activity is mediated by binding of the antibody molecule to the FcγRIII receptor, which has been shown to be dependent on the carbohydrate structure of the N-linked glycosylation at the N297 residue of the CH2 domain. Non-fucosylated antibodies bind this receptor with increased affinity and trigger FcγRIII-mediated effector functions more efficiently than native, fucosylated antibodies. For example, recombinant production of non-fucosylated antibody in CHO cells in which the alpha-1,6-fucosyl transferase enzyme has been knocked out results in antibody with 100-fold increased ADCC activity (see Yamane-Ohnuki et al., Biotechnol Bioeng. 87(5):614-22, 2004). Similar effects can be accomplished through decreasing the activity of alpha-1,6-fucosyl transferase enzyme or other enzymes in the fucosylation pathway, e.g., through siRNA or antisense RNA treatment, engineering cell lines to knockout the enzyme(s), or culturing with selective glycosylation inhibitors (see Rothman et al., Mol Immunol. 26(12):1113-23, 1989). Some host cell strains, e.g. Lec13 or rat hybridoma YB2/0 cell line naturally produce antibodies with lower fucosylation levels (see Shields et al., J Biol Chem. 277(30):26733-40, 2002 and Shinkawa et al., J Biol Chem. 278(5):3466-73, 2003). An increase in the level of bisected carbohydrate, e.g. through recombinantly producing antibody in cells that overexpress GnTIII enzyme, has also been determined to increase ADCC activity (see Umana et al., Nat Biotechnol. 17(2):176-80, 1999).

In other embodiments, glycosylation of the multispecific antibody constructs described herein is decreased or eliminated by removing one or more glycosylation sites, e.g., from the Fc region of the binding protein. Amino acid substitutions that eliminate or alter N-linked glycosylation sites can reduce or eliminate N-linked glycosylation of the antigen binding protein. In certain embodiments, the multispecific antibody constructs described herein comprise a mutation at position N297 (EU numbering), such as N297Q, N297A, or N297G. In one particular embodiment, the multispecific antibody constructs of the invention comprise a Fc region from a human IgG1 antibody with a N297G mutation. To improve the stability of molecules comprising a N297 mutation, the Fc region of the molecules may be further engineered. For instance, in some embodiments, one or more amino acids in the Fc region are substituted with cysteine to promote disulfide bond formation in the dimeric state. Residues corresponding to V259, A287, R292, V302, L306, V323, or 1332 (EU numbering) of an IgG1 Fc region may thus be substituted with cysteine. In one embodiment, specific pairs of residues are substituted with cysteine such that they preferentially form a disulfide bond with each other, thus limiting or preventing disulfide bond scrambling. In certain embodiments pairs include, but are not limited to, A287C and L306C, V259C and L306C, R292C and V302C, and V323C and I332C. In particular embodiments, the multispecific antibody constructs described herein comprise a Fc region from a human IgG1 antibody with mutations at R292C and V302C. In such embodiments, the Fc region may also comprise a N297G mutation. In certain embodiments, the multispecific antibody constructs described herein comprise a Fc region from a human IgG1 antibody with mutations at L234A and L235A. In particular embodiments, the multispecific antibody constructs described herein comprise a Fc region from a human IgG1 antibody with mutations at N297G, R292C, V302C, L234A, and L235A.

Modifications of the multispecific antibody constructs of the invention to increase serum half-life also may desirable, for example, by incorporation of or addition of a salvage receptor binding epitope (e.g., by mutation of the appropriate region or by incorporating the epitope into a peptide tag that is then fused to the antigen binding protein at either end or in the middle, e.g., by DNA or peptide synthesis; see, e.g., WO96/32478) or adding molecules such as PEG or other water soluble polymers, including polysaccharide polymers. The salvage receptor binding epitope preferably constitutes a region wherein any one or more amino acid residues from one or two loops of a Fc region are transferred to an analogous position in the antigen binding protein. In one embodiment, three or more residues from one or two loops of the Fc region are transferred. In one embodiment, the epitope is taken from the CH2 domain of the Fc region (e.g., an IgG Fc region) and transferred to the CH1, CH3, or VH region, or more than one such region, of the antigen binding protein. Alternatively, the epitope is taken from the CH2 domain of the Fc region and transferred to the CL region or VL region, or both, of the antigen binding protein. See International applications WO 97/34631 and WO 96/32478 for a description of Fc variants and their interaction with the salvage receptor.

The present invention includes one or more isolated nucleic acids encoding the multispecific antibody constructs and components thereof described herein. Nucleic acid molecules of the invention include DNA and RNA in both single-stranded and double-stranded form, as well as the corresponding complementary sequences. DNA includes, for example, cDNA, genomic DNA, chemically synthesized DNA, DNA amplified by PCR, and combinations thereof. The nucleic acid molecules of the invention include full-length genes or cDNA molecules as well as a combination of fragments thereof. In one embodiment, the nucleic acids of the invention are derived from human sources, but the invention includes those derived from non-human species, as well.

Relevant amino acid sequences from an immunoglobulin or region thereof (e.g. variable region, Fc region, etc.) or polypeptide of interest may be determined by direct protein sequencing, and suitable encoding nucleotide sequences can be designed according to a universal codon table. Alternatively, genomic or cDNA encoding monoclonal antibodies from which the binding domains of the multispecific antibody constructs of the invention may be derived can be isolated and sequenced from cells producing such antibodies using conventional procedures (e.g., by using oligonucleotide probes that are capable of binding specifically to genes encoding the heavy and light chains of the monoclonal antibodies).

An “isolated nucleic acid,” which is used interchangeably herein with “isolated polynucleotide,” is a nucleic acid that has been separated from adjacent genetic sequences present in the genome of the organism from which the nucleic acid was isolated, in the case of nucleic acids isolated from naturally-occurring sources. In the case of nucleic acids synthesized enzymatically from a template or chemically, such as PCR products, cDNA molecules, or oligonucleotides for example, it is understood that the nucleic acids resulting from such processes are isolated nucleic acids. An isolated nucleic acid molecule refers to a nucleic acid molecule in the form of a separate fragment or as a component of a larger nucleic acid construct. In one embodiment, the nucleic acids are substantially free from contaminating endogenous material. The nucleic acid molecule has been derived from DNA or RNA isolated at least once in substantially pure form and in a quantity or concentration enabling identification, manipulation, and recovery of its component nucleotide sequences by standard biochemical methods (such as those outlined in Sambrook et al., Molecular Cloning: A Laboratory Manual, 2nd ed., Cold Spring Harbor Laboratory, Cold Spring Harbor, N.Y. (1989)). Such sequences are provided and/or constructed in the form of an open reading frame uninterrupted by internal non-translated sequences, or introns, that are typically present in eukaryotic genes. Sequences of non-translated DNA can be present 5′ or 3′ from an open reading frame, where the same do not interfere with manipulation or expression of the coding region. Unless specified otherwise, the left-hand end of any single-stranded polynucleotide sequence discussed herein is the 5′ end; the left-hand direction of double-stranded polynucleotide sequences is referred to as the 5′ direction. The direction of 5′ to 3′ production of nascent RNA transcripts is referred to as the transcription direction; sequence regions on the DNA strand having the same sequence as the RNA transcript that are 5′ to the 5′ end of the RNA transcript are referred to as “upstream sequences;” sequence regions on the DNA strand having the same sequence as the RNA transcript that are 3′ to the 3′ end of the RNA transcript are referred to as “downstream sequences.”

The present invention also includes nucleic acids that hybridize under moderately stringent conditions, and highly stringent conditions, to nucleic acids encoding polypeptides as described herein. The basic parameters affecting the choice of hybridization conditions and guidance for devising suitable conditions are set forth by Sambrook, Fritsch, and Maniatis (1989, Molecular Cloning: A Laboratory Manual, Cold Spring Harbor Laboratory Press, Cold Spring Harbor, N.Y., chapters 9 and 11; and Current Protocols in Molecular Biology, 1995, Ausubel et al., eds., John Wiley & Sons, Inc., sections 2.10 and 6.3-6.4), and can be readily determined by those having ordinary skill in the art based on, for example, the length and/or base composition of the DNA. One way of achieving moderately stringent conditions involves the use of a prewashing solution containing 5×SSC, 0.5% SDS, 1.0 mM EDTA (pH 8.0), hybridization buffer of about 50% formamide, 6×SSC, and a hybridization temperature of about 55° C. (or other similar hybridization solutions, such as one containing about 50% formamide, with a hybridization temperature of about 42° C.), and washing conditions of about 60° C., in 0.5×SSC, 0.1% SDS. Generally, highly stringent conditions are defined as hybridization conditions as above, but with washing at approximately 68° C., 0.2×SSC, 0.1% SDS. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes after hybridization is complete. It should be understood that the wash temperature and wash salt concentration can be adjusted as necessary to achieve a desired degree of stringency by applying the basic principles that govern hybridization reactions and duplex stability, as known to those skilled in the art and described further below (see, e.g., Sambrook et al., 1989). When hybridizing a nucleic acid to a target nucleic acid of unknown sequence, the hybrid length is assumed to be that of the hybridizing nucleic acid. When nucleic acids of known sequence are hybridized, the hybrid length can be determined by aligning the sequences of the nucleic acids and identifying the region or regions of optimal sequence complementarity. The hybridization temperature for hybrids anticipated to be less than 50 base pairs in length should be 5 to 10° C. less than the melting temperature (Tm) of the hybrid, where Tm is determined according to the following equations. For hybrids less than 18 base pairs in length, Tm (° C.)=2(# of A+T bases)+4(# of G+C bases). For hybrids above 18 base pairs in length, Tm (° C.)=81.5+16.6(log 10 [Na+])+0.41(% G+C)−(600/N), where N is the number of bases in the hybrid, and [Na+] is the concentration of sodium ions in the hybridization buffer ([Na+] for 1×SSC=0.165M). In one embodiment, each such hybridizing nucleic acid has a length that is at least 15 nucleotides (or at least 18 nucleotides, or at least 20 nucleotides, or at least 25 nucleotides, or at least 30 nucleotides, or at least 40 nucleotides, or at least 50 nucleotides), or at least 25% (or at least 50%, or at least 60%, or at least 70%, or at least 80%) of the length of the nucleic acid of the present invention to which it hybridizes, and has at least 60% sequence identity (or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or at least 99.5%) with the nucleic acid of the present invention to which it hybridizes, where sequence identity is determined by comparing the sequences of the hybridizing nucleic acids when aligned so as to maximize overlap and identity while minimizing sequence gaps as described in more detail above.

Variants of the antigen binding proteins described herein can be prepared by site-specific mutagenesis of nucleotides in the DNA encoding the polypeptide, using cassette or PCR mutagenesis or other techniques well known in the art, to produce DNA encoding the variant, and thereafter expressing the recombinant DNA in cell culture as outlined herein. However, antigen binding proteins comprising variant CDRs having up to about 100-150 residues may be prepared by in vitro synthesis using established techniques. The variants typically exhibit the same qualitative biological activity as the naturally occurring analogue, e.g., binding to antigen. Such variants include, for example, deletions and/or insertions and/or substitutions of residues within the amino acid sequences of the antigen binding proteins.

Any combination of deletion, insertion, and substitution is made to arrive at the final construct, provided that the final construct possesses the desired characteristics. The amino acid changes also may alter post-translational processes of the antigen binding protein, such as changing the number or position of glycosylation sites. In certain embodiments, antigen binding protein variants are prepared with the intent to modify those amino acid residues which are directly involved in epitope binding. In other embodiments, modification of residues which are not directly involved in epitope binding or residues not involved in epitope binding in any way, is desirable, for purposes discussed herein. Mutagenesis within any of the CDR regions and/or framework regions is contemplated. Covariance analysis techniques can be employed by the skilled artisan to design useful modifications in the amino acid sequence of the antigen binding protein. See, e.g., Choulier, et al., Proteins 41:475-484, 2000; Demarest et al., J. Mol. Biol. 335:41-48, 2004; Hugo et al., Protein Engineering 16(5):381-86, 2003; Aurora et al., US Patent Publication No. 2008/0318207 A1; Glaser et al., US Patent Publication No. 2009/0048122 A1; Urech et al., WO 2008/110348 A1; Borras et al., WO 2009/000099 A2. Such modifications determined by covariance analysis can improve potency, pharmacokinetic, pharmacodynamic, and/or manufacturability characteristics of an antigen binding protein.

The nucleic acid sequences of the present invention. As will be appreciated by those in the art, due to the degeneracy of the genetic code, an extremely large number of nucleic acids may be made, all of which encode the CDRs (and heavy and light chains or other components of the antigen binding proteins described herein) of the invention. Thus, having identified a particular amino acid sequence, those skilled in the art could make any number of different nucleic acids, by simply modifying the sequence of one or more codons in a way which does not change the amino acid sequence of the encoded protein.

The present invention also includes vectors comprising one or more nucleic acids encoding one or more components of the multispecific antibody constructs of the invention (e.g. variable regions, light chains, heavy chains, modified heavy chains, and Fd fragments). The term “vector” refers to any molecule or entity (e.g., nucleic acid, plasmid, bacteriophage or virus) used to transfer protein coding information into a host cell. Examples of vectors include, but are not limited to, plasmids, viral vectors, non-episomal mammalian vectors and expression vectors, for example, recombinant expression vectors. The term “expression vector” or “expression construct” as used herein refers to a recombinant DNA molecule containing a desired coding sequence and appropriate nucleic acid control sequences necessary for the expression of the operably linked coding sequence in a particular host cell. An expression vector can include, but is not limited to, sequences that affect or control transcription, translation, and, if introns are present, affect RNA splicing of a coding region operably linked thereto. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals. A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired. For instance, in some embodiments, signal peptide sequences may be appended/fused to the amino terminus of any of the polypeptides sequences of the present invention. In certain embodiments, a signal peptide having the amino acid sequence of MDMRVPAQLLGLLLLWLRGARC (SEQ ID NO: 32) is fused to the amino terminus of any of the polypeptide sequences of the present invention. In other embodiments, a signal peptide having the amino acid sequence of MAWALLLLTLLTQGTGSWA (SEQ ID NO: 33) is fused to the amino terminus of any of the polypeptide sequences of the present invention. In still other embodiments, a signal peptide having the amino acid sequence of MTCSPLLLTLLIHCTGSWA (SEQ ID NO: 34) is fused to the amino terminus of any of the polypeptide sequences of the present invention. Other suitable signal peptide sequences that can be fused to the amino terminus of the polypeptide sequences described herein include: MEAPAQLLFLLLLWLPDTTG (SEQ ID NO: 35), MEWTWRVLFLVAAATGAHS (SEQ ID NO: 36), METPAQLLFLLLLWLPDTTG (SEQ ID NO: 37), MKHLWFFLLLVAAPRWVLS (SEQ ID NO: 38), and MEWSWVFLFFLSVTTGVHS (SEQ ID NO: 39). Other signal peptides are known to those of skill in the art and may be fused to any of the polypeptide chains of the present invention, for example, to facilitate or optimize expression in particular host cells.

Typically, expression vectors used in the host cells to produce the bispecific antigen proteins of the invention will contain sequences for plasmid maintenance and for cloning and expression of exogenous nucleotide sequences encoding the components of the bispecific antigen binding proteins. Such sequences, collectively referred to as “flanking sequences,” in certain embodiments will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Each of these sequences is discussed below.

Optionally, the vector may contain a “tag”-encoding sequence, i.e., an oligonucleotide molecule located at the 5′ or 3′ end of the polypeptide coding sequence; the oligonucleotide tag sequence encodes polyHis (such as hexaHis), FLAG, HA (hemaglutinin influenza virus), myc, or another “tag” molecule for which commercially available antibodies exist. This tag is typically fused to the polypeptide upon expression of the polypeptide, and can serve as a means for affinity purification or detection of the polypeptide from the host cell. Affinity purification can be accomplished, for example, by column chromatography using antibodies against the tag as an affinity matrix. Optionally, the tag can subsequently be removed from the purified polypeptide by various means such as using certain peptidases for cleavage.

Flanking sequences may be homologous (i.e., from the same species and/or strain as the host cell), heterologous (i.e., from a species other than the host cell species or strain), hybrid (i.e., a combination of flanking sequences from more than one source), synthetic or native. As such, the source of a flanking sequence may be any prokaryotic or eukaryotic organism, any vertebrate or invertebrate organism, or any plant, provided that the flanking sequence is functional in, and can be activated by, the host cell machinery.

Flanking sequences useful in the vectors of this invention may be obtained by any of several methods well known in the art. Typically, flanking sequences useful herein will have been previously identified by mapping and/or by restriction endonuclease digestion and can thus be isolated from the proper tissue source using the appropriate restriction endonucleases. In some cases, the full nucleotide sequence of a flanking sequence may be known. Here, the flanking sequence may be synthesized using routine methods for nucleic acid synthesis or cloning.

Whether all or only a portion of the flanking sequence is known, it may be obtained using polymerase chain reaction (PCR) and/or by screening a genomic library with a suitable probe such as an oligonucleotide and/or flanking sequence fragment from the same or another species. Where the flanking sequence is not known, a fragment of DNA containing a flanking sequence may be isolated from a larger piece of DNA that may contain, for example, a coding sequence or even another gene or genes. Isolation may be accomplished by restriction endonuclease digestion to produce the proper DNA fragment followed by isolation using agarose gel purification, Qiagen® column chromatography (Chatsworth, Calif.), or other methods known to the skilled artisan. The selection of suitable enzymes to accomplish this purpose will be readily apparent to one of ordinary skill in the art.

An origin of replication is typically a part of those prokaryotic expression vectors purchased commercially, and the origin aids in the amplification of the vector in a host cell. If the vector of choice does not contain an origin of replication site, one may be chemically synthesized based on a known sequence, and ligated into the vector. For example, the origin of replication from the plasmid pBR322 (New England Biolabs, Beverly, Mass.) is suitable for most gram-negative bacteria, and various viral origins (e.g., SV40, polyoma, adenovirus, vesicular stomatitus virus (VSV), or papillomaviruses such as HPV or BPV) are useful for cloning vectors in mammalian cells. Generally, the origin of replication component is not needed for mammalian expression vectors (for example, the SV40 origin is often used only because it also contains the virus early promoter).

A transcription termination sequence is typically located 3′ to the end of a polypeptide coding region and serves to terminate transcription. Usually, a transcription termination sequence in prokaryotic cells is a G-C rich fragment followed by a poly-T sequence. While the sequence is easily cloned from a library or even purchased commercially as part of a vector, it can also be readily synthesized using known methods for nucleic acid synthesis.

A selectable marker gene encodes a protein necessary for the survival and growth of a host cell grown in a selective culture medium. Typical selection marker genes encode proteins that (a) confer resistance to antibiotics or other toxins, e.g., ampicillin, tetracycline, or kanamycin for prokaryotic host cells; (b) complement auxotrophic deficiencies of the cell; or (c) supply critical nutrients not available from complex or defined media. Specific selectable markers are the kanamycin resistance gene, the ampicillin resistance gene, and the tetracycline resistance gene. Advantageously, a neomycin resistance gene may also be used for selection in both prokaryotic and eukaryotic host cells.

Other selectable genes may be used to amplify the gene that will be expressed. Amplification is the process wherein genes that are required for production of a protein critical for growth or cell survival are reiterated in tandem within the chromosomes of successive generations of recombinant cells. Examples of suitable selectable markers for mammalian cells include dihydrofolate reductase (DHFR) and promoterless thymidine kinase genes. Mammalian cell transformants are placed under selection pressure wherein only the transformants are uniquely adapted to survive by virtue of the selectable gene present in the vector. Selection pressure is imposed by culturing the transformed cells under conditions in which the concentration of selection agent in the medium is successively increased, thereby leading to the amplification of both the selectable gene and the DNA that encodes another gene, such as one or more components of the multispecific antibody constructs described herein. As a result, increased quantities of a polypeptide are synthesized from the amplified DNA.

A ribosome-binding site is usually necessary for translation initiation of mRNA and is characterized by a Shine-Dalgarno sequence (prokaryotes) or a Kozak sequence (eukaryotes). The element is typically located 3′ to the promoter and 5′ to the coding sequence of the polypeptide to be expressed. In certain embodiments, one or more coding regions may be operably linked to an internal ribosome binding site (IRES), allowing translation of two open reading frames from a single RNA transcript.

In some cases, such as where glycosylation is desired in a eukaryotic host cell expression system, one may manipulate the various pre- or prosequences to improve glycosylation or yield. For example, one may alter the peptidase cleavage site of a particular signal peptide, or add prosequences, which also may affect glycosylation. The final protein product may have, in the −1 position (relative to the first amino acid of the mature protein) one or more additional amino acids incident to expression, which may not have been totally removed. For example, the final protein product may have one or two amino acid residues found in the peptidase cleavage site, attached to the amino-terminus. Alternatively, use of some enzyme cleavage sites may result in a slightly truncated form of the desired polypeptide, if the enzyme cuts at such area within the mature polypeptide.

Expression and cloning vectors of the invention will typically contain a promoter that is recognized by the host organism and operably linked to the molecule encoding the polypeptide. The term “operably linked” as used herein refers to the linkage of two or more nucleic acid sequences in such a manner that a nucleic acid molecule capable of directing the transcription of a given gene and/or the synthesis of a desired protein molecule is produced. For example, a control sequence in a vector that is “operably linked” to a protein coding sequence is ligated thereto so that expression of the protein coding sequence is achieved under conditions compatible with the transcriptional activity of the control sequences. More specifically, a promoter and/or enhancer sequence, including any combination of cis-acting transcriptional control elements is operably linked to a coding sequence if it stimulates or modulates the transcription of the coding sequence in an appropriate host cell or other expression system.

Promoters are untranscribed sequences located upstream (i.e., 5′) to the start codon of a structural gene (generally within about 100 to 1000 bp) that control transcription of the structural gene. Promoters are conventionally grouped into one of two classes: inducible promoters and constitutive promoters. Inducible promoters initiate increased levels of transcription from DNA under their control in response to some change in culture conditions, such as the presence or absence of a nutrient or a change in temperature. Constitutive promoters, on the other hand, uniformly transcribe a gene to which they are operably linked, that is, with little or no control over gene expression. A large number of promoters, recognized by a variety of potential host cells, are well known. A suitable promoter is operably linked to the DNA encoding e.g., heavy chain, light chain, modified heavy chain, or other component of the multispecific antibody constructs of the invention, by removing the promoter from the source DNA by restriction enzyme digestion and inserting the desired promoter sequence into the vector.

Suitable promoters for use with yeast hosts are also well known in the art. Yeast enhancers are advantageously used with yeast promoters. Suitable promoters for use with mammalian host cells are well known and include, but are not limited to, those obtained from the genomes of viruses such as polyoma virus, fowlpox virus, adenovirus (such as Adenovirus 2), bovine papilloma virus, avian sarcoma virus, cytomegalovirus, retroviruses, hepatitis-B virus and Simian Virus 40 (SV40). Other suitable mammalian promoters include heterologous mammalian promoters, for example, heat-shock promoters and the actin promoter.

Additional promoters which may be of interest include, but are not limited to: SV40 early promoter (Benoist and Chambon, 1981, Nature 290:304-310); CMV promoter (Thomsen et al., 1984, Proc. Natl. Acad. U.S.A. 81:659-663); the promoter contained in the 3′ long terminal repeat of Rous sarcoma virus (Yamamoto et al., 1980, Cell 22:787-797); herpes thymidine kinase promoter (Wagner et al., 1981, Proc. Natl. Acad. Sci. U.S.A. 78: 1444-1445); promoter and regulatory sequences from the metallothionine gene Prinster et al., 1982, Nature 296:39-42); and prokaryotic promoters such as the beta-lactamase promoter (Villa-Kamaroff et al., 1978, Proc. Natl. Acad. Sci. U.S.A. 75:3727-3731); or the tac promoter (DeBoer et al., 1983, Proc. Natl. Acad. Sci. U.S.A. 80:21-25). Also of interest are the following animal transcriptional control regions, which exhibit tissue specificity and have been utilized in transgenic animals: the elastase I gene control region that is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); the insulin gene control region that is active in pancreatic beta cells (Hanahan, 1985, Nature 315: 115-122); the immunoglobulin gene control region that is active in lymphoid cells (Grosschedl et al., 1984, Cell 38:647-658; Adames et al., 1985, Nature 318:533-538; Alexander et al., 1987, Mol. Cell. Biol. 7: 1436-1444); the mouse mammary tumor virus control region that is active in testicular, breast, lymphoid and mast cells (Leder et al., 1986, Cell 45:485-495); the albumin gene control region that is active in liver (Pinkert et al., 1987, Genes and Devel. 1:268-276); the alpha-feto-protein gene control region that is active in liver (Krumlauf et al., 1985, Mol. Cell. Biol. 5: 1639-1648; Hammer et al., 1987, Science 253:53-58); the alpha 1-antitrypsin gene control region that is active in liver (Kelsey et al., 1987, Genes and Devel. 1: 161-171); the beta-globin gene control region that is active in myeloid cells (Mogram et al, 1985, Nature 315:338-340; Kollias et al, 1986, Cell 46:89-94); the myelin basic protein gene control region that is active in oligodendrocyte cells in the brain (Readhead et al., 1987, Cell 48:703-712); the myosin light chain-2 gene control region that is active in skeletal muscle (Sani, 1985, Nature 314:283-286); and the gonadotropic releasing hormone gene control region that is active in the hypothalamus (Mason et al., 1986, Science 234: 1372-1378).

An enhancer sequence may be inserted into the vector to increase transcription of DNA encoding a component of the multispecific antibody constructs (e.g., light chain, heavy chain, modified heavy chain, Fd fragment) by higher eukaryotes. Enhancers are cis-acting elements of DNA, usually about 10-300 bp in length, that act on the promoter to increase transcription. Enhancers are relatively orientation and position independent, having been found at positions both 5′ and 3′ to the transcription unit. Several enhancer sequences available from mammalian genes are known (e.g., globin, elastase, albumin, alpha-feto-protein and insulin). Typically, however, an enhancer from a virus is used. The SV40 enhancer, the cytomegalovirus early promoter enhancer, the polyoma enhancer, and adenovirus enhancers known in the art are exemplary enhancing elements for the activation of eukaryotic promoters. While an enhancer may be positioned in the vector either 5′ or 3′ to a coding sequence, it is typically located at a site 5′ from the promoter. A sequence encoding an appropriate native or heterologous signal sequence (leader sequence or signal peptide) can be incorporated into an expression vector, to promote extracellular secretion of the antibody. The choice of signal peptide or leader depends on the type of host cells in which the antibody is to be produced, and a heterologous signal sequence can replace the native signal sequence. Examples of signal peptides are described above. Other signal peptides that are functional in mammalian host cells include the signal sequence for interleukin-7 (IL-7) described in U.S. Pat. No. 4,965,195; the signal sequence for interleukin-2 receptor described in Cosman et al., 1984, Nature 312:768; the interleukin-4 receptor signal peptide described in EP Patent No. 0367 566; the type I interleukin-1 receptor signal peptide described in U.S. Pat. No. 4,968,607; the type II interleukin-1 receptor signal peptide described in EP Patent No. 0 460 846.

The expression vectors that are provided may be constructed from a starting vector such as a commercially available vector. Such vectors may or may not contain all of the desired flanking sequences. Where one or more of the flanking sequences described herein are not already present in the vector, they may be individually obtained and ligated into the vector. Methods used for obtaining each of the flanking sequences are well known to one skilled in the art. The expression vectors can be introduced into host cells to thereby produce proteins, including fusion proteins, encoded by nucleic acids as described herein.

In certain embodiments, nucleic acids encoding the different components of the multispecific antibody constructs of the invention may be inserted into the same expression vector. For instance, the nucleic acid encoding an anti-first target antigen light chain can be cloned into the same vector as the nucleic acid encoding an anti-first target antigen heavy chain. In such embodiments, the two nucleic acids may be separated by an internal ribosome entry site (IRES) and under the control of a single promoter such that the light chain and heavy chain are expressed from the same mRNA transcript. Alternatively, the two nucleic acids may be under the control of two separate promoters such that the light chain and heavy chain are expressed from two separate mRNA transcripts. In some embodiments, nucleic acids encoding the anti-first target antigen light chain and heavy chain are cloned into one expression vector and the nucleic acids encoding the anti-second target antigen light chain and heavy chain are cloned into a second expression vector.

After the vector has been constructed and the one or more nucleic acid molecules encoding the components of the multispecific antibody constructs described herein has been inserted into the proper site(s) of the vector or vectors, the completed vector(s) may be inserted into a suitable host cell for amplification and/or polypeptide expression. Thus, the present invention encompasses an isolated host cell comprising one or more expression vectors encoding the components of the bispecific antigen binding proteins. The term “host cell” as used herein refers to a cell that has been transformed, or is capable of being transformed, with a nucleic acid and thereby expresses a gene of interest. The term includes the progeny of the parent cell, whether or not the progeny is identical in morphology or in genetic make-up to the original parent cell, so long as the gene of interest is present. A host cell that comprises an isolated nucleic acid of the invention, in one embodiment operably linked to at least one expression control sequence (e.g. promoter or enhancer), is a “recombinant host cell.”

The transformation of an expression vector for an antigen binding protein into a selected host cell may be accomplished by well-known methods including transfection, infection, calcium phosphate co-precipitation, electroporation, microinjection, lipofection, DEAE-dextran mediated transfection, or other known techniques. The method selected will in part be a function of the type of host cell to be used. These methods and other suitable methods are well known to the skilled artisan, and are set forth, for example, in Sambrook et al., 2001, supra.

A host cell, when cultured under appropriate conditions, synthesizes an antigen binding protein that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule.

Exemplary host cells include prokaryote, yeast, or higher eukaryote cells. Prokaryotic host cells include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacillus, such as B. subtilis and B. licheniformis, Pseudomonas, and Streptomyces. Eukaryotic microbes such as filamentous fungi or yeast are suitable cloning or expression hosts for recombinant polypeptides. Saccharomyces cerevisiae, or common baker's yeast, is the most commonly used among lower eukaryotic host microorganisms. However, a number of other genera, species, and strains are commonly available and useful herein, such as Pichia, e.g. P. pastoris, Schizosaccharomyces pombe; Kluyveromyces, Yarrowia; Candida; Trichoderma reesia; Neurospora crassa; Schwanniomyces, such as Schwanniomyces occidentalis; and filamentous fungi, such as, e.g., Neurospora, Penicillium, Tolypocladium, and Aspergillus hosts such as A. nidulans and A. niger.

Host cells for the expression of glycosylated antigen binding proteins can be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection of such cells are publicly available, e.g., the L-1 variant of Autographa californica NPV and the Bm-5 strain of Bombyx mori NPV.

Vertebrate host cells are also suitable hosts, and recombinant production of antigen binding proteins from such cells has become routine procedure. Mammalian cell lines available as hosts for expression are well known in the art and include, but are not limited to, immortalized cell lines available from the American Type Culture Collection (ATCC), including but not limited to Chinese hamster ovary (CHO) cells, including CHOK1 cells (ATCC CCL61), DXB-11, DG-44, and Chinese hamster ovary cells/-DHFR (CHO, Urlaub et al., Proc. Natl. Acad. Sci. USA 77: 4216, 1980); monkey kidney CV1 line transformed by SV40 (COS-7, ATCC CRL 1651); human embryonic kidney line (293 or 293 cells subcloned for growth in suspension culture, (Graham et al., J. Gen Virol. 36: 59, 1977); baby hamster kidney cells (BHK, ATCC CCL 10); mouse sertoli cells (TM4, Mather, Biol. Reprod. 23: 243-251, 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TRI cells (Mather et al., Annals N.Y Acad. Sci. 383: 44-68, 1982); MRC 5 cells or FS4 cells; mammalian myeloma cells, and a number of other cell lines. In certain embodiments, cell lines may be selected through determining which cell lines have high expression levels and constitutively produce multispecific antibody constructs of the present invention. In another embodiment, a cell line from the B cell lineage that does not make its own antibody but has a capacity to make and secrete a heterologous antibody can be selected. CHO cells are host cells in some embodiments for expressing the multispecific antibody constructs of the invention.

Host cells are transformed or transfected with the above-described nucleic acids or vectors for production of multispecific antibody constructs and are cultured in conventional nutrient media modified as appropriate for inducing promoters, selecting transformants, or amplifying the genes encoding the desired sequences. In addition, novel vectors and transfected cell lines with multiple copies of transcription units separated by a selective marker are particularly useful for the expression of antigen binding proteins. Thus, the present invention also provides a method for preparing a bispecific antigen binding protein described herein comprising culturing a host cell comprising one or more expression vectors described herein in a culture medium under conditions permitting expression of the bispecific antigen binding protein encoded by the one or more expression vectors; and recovering the bispecific antigen binding protein from the culture medium.

The host cells used to produce the antigen binding proteins of the invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58: 44, 1979; Barnes et al., Anal. Biochem. 102: 255, 1980; U.S. Pat. Nos. 4,767,704; 4,657,866; 4,927,762; 4,560,655; or 5,122,469; WO90103430; WO 87/00195; or U.S. Pat. Re. No. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Upon culturing the host cells, the bispecific antigen binding protein can be produced intracellularly, in the periplasmic space, or directly secreted into the medium. If the antigen binding protein is produced intracellularly, as a first step, the particulate debris, either host cells or lysed fragments, is removed, for example, by centrifugation or ultrafiltration. The bispecifc antigen binding protein can be purified using, for example, hydroxyapatite chromatography, cation or anion exchange chromatography, or affinity chromatography, using the antigen(s) of interest or protein A or protein G as an affinity ligand. Protein A can be used to purify proteins that include polypeptides that are based on human γ1, γ2, or γ4 heavy chains (Lindmark et al., J. Immunol. Meth. 62: 1-13, 1983). Protein G is recommended for all mouse isotypes and for human γ3 (Guss et al., EMBO J. 5: 15671575, 1986). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the protein comprises a CH3 domain, the Bakerbond ABX™ resin (J. T. Baker, Phillipsburg, N.J.) is useful for purification. Other techniques for protein purification such as ethanol precipitation, Reverse Phase HPLC, chromatofocusing, SDS-PAGE, and ammonium sulfate precipitation are also possible depending on the particular bispecific antigen binding protein to be recovered.

### Examples

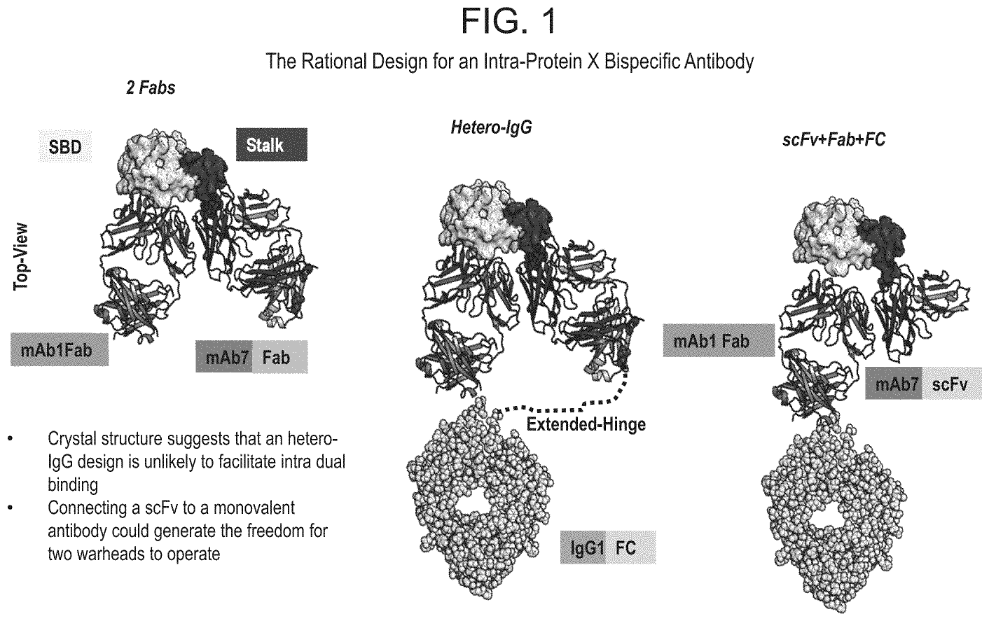

The rational to design this novel format of bispecifics that can be widely used in both cis and trans mechanisms of action took place upon the determination of a crystal structure of ternary complex with 2 Fabs binding 2 different domains in the same target receptor molecule. This provided a template to conceptualize a linker connecting those 2 molecules and making them a single molecule upon recombinant expression in a single cell (FIG. 1). The use of this linker to connect these 2 warheads is important to enforce a specific binding mode that can be tailored to specific needs of specific therapeutic projects.

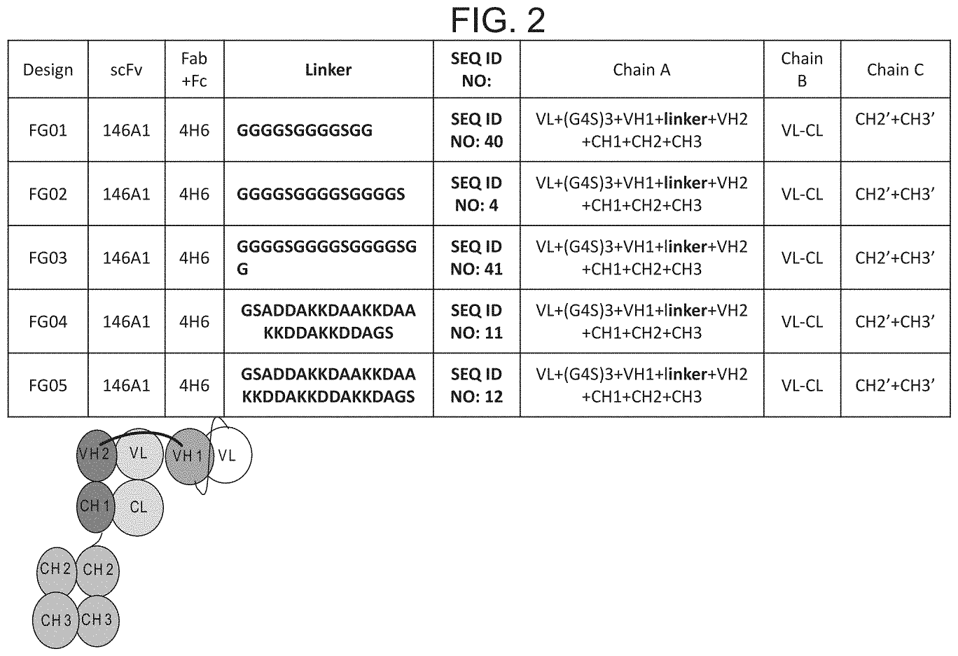

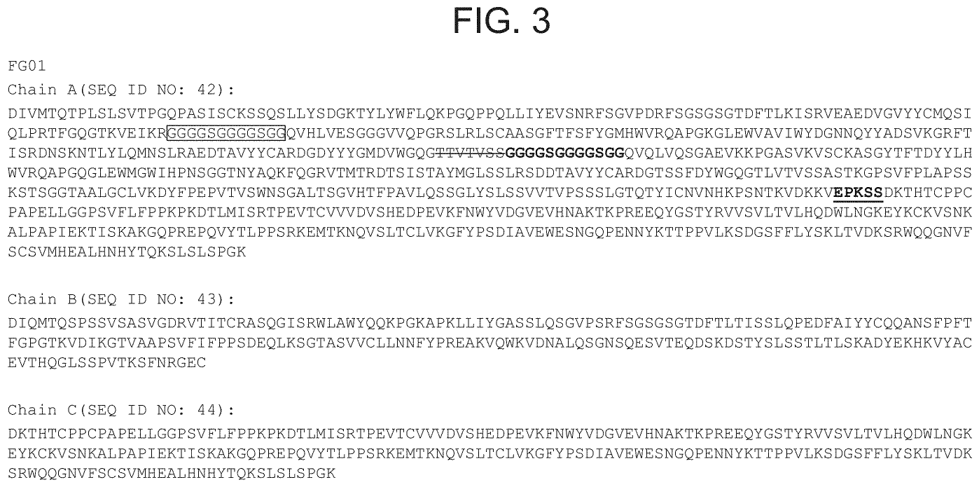

In order to attach an scFv module to a Fab, three flexible G4S linkers with multiple repeats and two semi-rigid helical linkers with different lengths (FIG. 2) have been engineered. To increase the half life of the molecule, an FC region at the C-terminus of the Fab CH1 domain (FIGS. 2 and 3) was inserted, resulting in a molecule with 3 polypeptide chains.

Alternatively, the scFv can also be connected to the N-terminus of the light chain in the Fab as well. This can allow the molecule design to meet specific needs of the binding mode required and also balance the length of the polypeptide chains that comprise the molecule (FIG. 3).

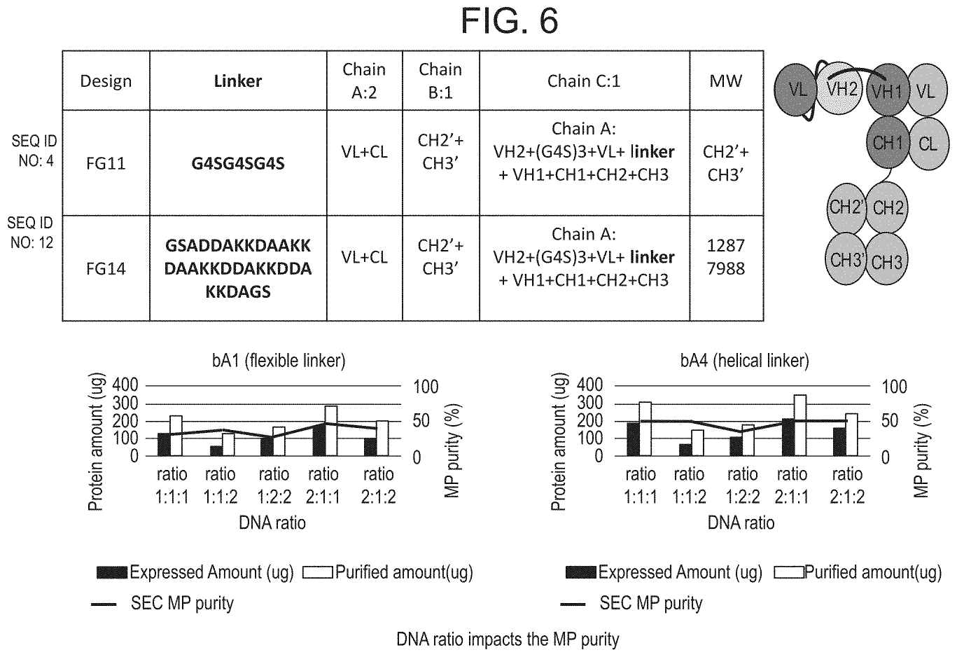

The molecules were expressed in HEK 293 6E cells and purified for ProA with total yields around 75 mg/L (FIG. 6).

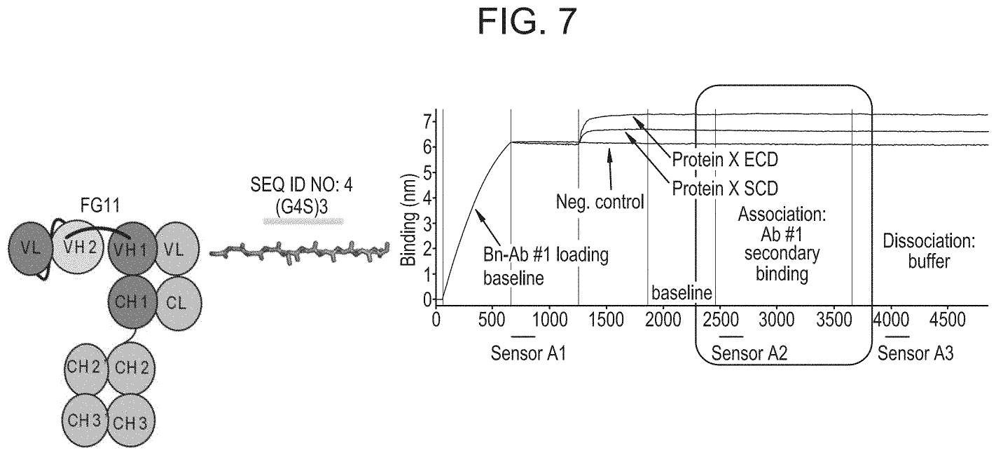

The molecules were then tested for binding assays where the binding to the same receptor from both warheads was strictly enforced. FG11 met the design goal and it did not recognize/bind to epitopes in 2 different receptor molecules (FIG. 7).

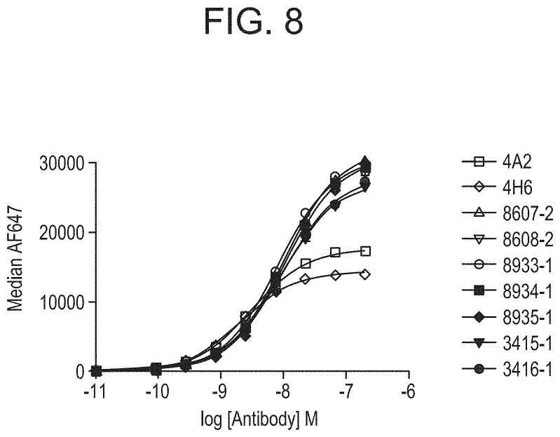

To confirm that these bispecific molecules were also functional while binding to its target on the surface of a cell, a cell-based assay expressing the human target protein was performed. In this case all bispecific molecules showed binding (FIG. 8).

All publications, patents, and patent applications discussed and cited herein are hereby incorporated by reference in their entireties. It is understood that the disclosed invention is not limited to the particular methodology, protocols and materials described as these can vary. It is also understood that the terminology used herein is for the purposes of describing particular embodiments only and is not intended to limit the scope of the appended claims.

Those skilled in the art will recognize, or be able to ascertain using no more than routine experimentation, many equivalents to the specific embodiments of the invention described herein. Such equivalents are intended to be encompassed by the following claims.

What is claimed is:

1. A multispecific antibody construct comprising:
a) a first polypeptide comprising an antibody Fc region, the antibody Fc region comprising a first hinge region, a first CH2 region, and a first CH3 region;
b) a second polypeptide comprising an antibody heavy chain construct, the antibody heavy chain construct comprising
i) a scFv, the scFv comprising
1) a first VH and a first VL,
wherein the first VH and the first VL associate to form a first antigen binding domain, and
2) a first linker peptide that connects the first VH and first VL; and
ii) an antibody heavy chain, the antibody heavy chain comprising a second VH, a second CH1 region, a second hinge region, a second CH2 region, and a second CH3 region;
wherein the scFv is attached at its C-terminus to the N-terminus of the second VH region of the antibody heavy chain;
c) a third polypeptide comprising an antibody light chain comprising a second VL and a CL,
wherein the second VH of the antibody heavy chain and the second VL of the antibody light chain associate to form a second antigen binding domain.

2. The multispecific antibody construct of claim 1, wherein the scFv is attached to the antibody heavy chain via a second linker.

3. The multispecific antibody construct of claim 2, wherein the second linker comprises a sequence selected from the group consisting of: (Gly₃Ser)₂ (SEQ ID NO: 1), (Gly₄Ser)₂ (SEQ ID NO: 2), (Gly₃Ser)₃ (SEQ ID NO: 3), (Gly₄Ser)₃ (SEQ ID NO: 4), (Gly₃Ser)₄ (SEQ ID NO: 5), (Gly₄Ser)₄ (SEQ ID NO: 6), (Gly₃Ser)₅ (SEQ ID NO: 7), (Gly₄Ser)₅ (SEQ ID NO: 8), (Gly₃Ser)₆ (SEQ ID NO: 9), and (Gly₄Ser)₆ (SEQ ID NO: 10).

4. The multispecific antibody construct of claim 1, wherein the first linker comprises a sequence selected from the group consisting of: (Gly₃Ser)₂ (SEQ ID NO: 1), (Gly₄Ser)₂ (SEQ ID NO: 2), (Gly₃Ser)₃ (SEQ ID NO: 3), (Gly₄Ser)₃ (SEQ ID NO: 4), (Gly₃Ser)₄ (SEQ ID NO: 5), (Gly₄Ser)₄ (SEQ ID NO: 6), (Gly₃Ser)₅ (SEQ ID NO: 7), (Gly₄Ser)₅ (SEQ ID NO: 8), (Gly₃Ser)₆ (SEQ ID NO: 9), (Gly₄Ser)₆ (SEQ ID NO: 10), GSADDAKKDAAKKDAAKKDDAKKDDAGS (SEQ ID NO: 11), GSADDAKKDAAKKDAAKKDDAKKDDAKKDAGS (SEQ ID NO: 12), (Gly₃Gln)₂ (SEQ ID NO: 13), (Gly₄Gln)₂ (SEQ ID NO: 14), (Gly₃ Gln)₃ (SEQ ID NO: 15), (Gly₄Gln)₃ (SEQ ID NO: 16), (Gly₃Gln)₄ (SEQ ID NO: 17), (Gly₄Gln)₄ (SEQ ID NO: 18), (Gly₃Gln)₅ (SEQ ID NO: 19), (Gly₄Gln)₅ (SEQ ID NO: 20), (Gly₃Gln)₆ (SEQ ID NO: 21), and (Gly₄Gln)₆ (SEQ ID NO: 22).

5. The multispecific antibody construct of claim 1, wherein the scFv comprises the first VH attached at its C-terminus to the N-terminus of the first linker and the first linker is attached at its C-terminus to the N-terminus of the first VL.

6. The multispecific antibody construct of claim 1, wherein the scFv comprises the first VL attached at its C-terminus to the N-terminus of the first linker and the first linker is attached at its C-terminus to the N-terminus of the first VH.

7. The multispecific antibody construct of claim 1, wherein the first antigen binding domain and the second antigen binding domain bind to epitopes on different polypeptides.

8. The multispecific antibody construct of claim 1, wherein the first antigen binding domain and the second antigen binding domain bind to different epitopes on the same polypeptide.

9. The multispecific antibody construct of claim 8, wherein the multispecific antibody construct is a biparatopic antibody construct.

10. The multispecific antibody construct of claim 1, wherein the Fc region consists of a hinge region, CH2 region, and CH3 regions.

11. The multispecific antibody construct of claim 1, wherein the N-terminus of the Fc region is linked via its N-terminus to the C-terminus of the heavy chain via a third linker.

12. The multispecific antibody construct of claim 11, wherein the third linker comprises a sequence selected from the group consisting of: (Gly₃Ser)₂ (SEQ ID NO: 1), (Gly₄Ser)₂ (SEQ ID NO: 2), (Gly₃Ser)₃ (SEQ ID NO: 3), (Gly₄Ser)₃ (SEQ ID NO: 4), (Gly₃Ser)₄ (SEQ ID NO: 5), (Gly₄Ser)₄ (SEQ ID NO: 6), (Gly₃Ser)₅ (SEQ ID NO: 7), (Gly₄Ser)₅ (SEQ ID NO: 8), (Gly₃Ser)₆ (SEQ ID NO: 9), (Gly₄Ser)₆ (SEQ ID NO: 10), (Gly₃Gln)₂ (SEQ ID NO: 13), (Gly₄Gln)₂ (SEQ ID NO: 14), (Gly₃ Gln)₃ (SEQ ID NO: 15), (Gly₄Gln)₃ (SEQ ID NO: 16), (Gly₃Gln)₄ (SEQ ID NO: 17), (Gly₄Gln)₄ (SEQ ID NO: 18), (Gly₃Gln)₅ (SEQ ID NO: 19), (Gly₄Gln)₅ (SEQ ID NO: 20), (Gly₃Gln)₆ (SEQ ID NO: 21), and (Gly₄Gln)₆ (SEQ ID NO: 22).

13. The multispecific antibody construct of claim 1, wherein the first polypeptide consists of the antibody Fc region.

14. A multispecific antibody construct comprising:
a) a first polypeptide comprising an antibody Fc region, the antibody Fc region comprising a first hinge region, a first CH2 region, and first CH3 regions;
b) a second polypeptide comprising an antibody light chain construct, the antibody light chain construct comprising
i) a scFv, the scFv comprising
1) a first VH and a first VL,
wherein the first VH and the first VL associate to form a first antigen binding domain, and
2) a first linker peptide that connects the first VH and first VL; and
ii) an antibody light chain comprising a second VL and a CL;
wherein the scFv is attached at its C-terminus to the N-terminus of the second VL region of the antibody light chain;
c) a third polypeptide comprising an antibody heavy chain, the antibody heavy chain comprising a second VH, a second CH1 region, a second hinge region, a second CH2 region, and a second CH3 region,
wherein the second VH of the antibody heavy chain and the second VL of the antibody light chain associate to form a second antigen binding domain.

15. The multispecific antibody construct of claim 14, wherein the scFv is attached to the antibody light chain via a second linker.

16. The multispecific antibody construct of claim 15, wherein the second linker comprises a sequence selected from the group consisting of: (Gly₃Ser)₂ (SEQ ID NO: 1), (Gly₄Ser)₂ (SEQ ID NO: 2), (Gly₃Ser)₃ (SEQ ID NO: 3), (Gly₄Ser)₃ (SEQ ID NO: 4), (Gly₃Ser)₄ (SEQ ID NO: 5), (Gly₄Ser)₄ (SEQ ID NO: 6), (Gly₃Ser)₅ (SEQ ID NO: 7), (Gly₄Ser)₅ (SEQ ID NO: 8), (Gly₃Ser)₆ (SEQ ID NO: 9), (Gly₄Ser)₆ (SEQ ID NO: 10), GSADDAKKDAAKKDAAKKDDAKKDDAGS (SEQ ID NO: 11), GSADDAKKDAAKKDAAKKDDAKKDDAKKDAGS (SEQ ID NO: 12), (Gly₃Gln)₂ (SEQ ID NO: 13), (Gly₄Gln)₂ (SEQ ID NO: 14), (Gly₃ Gln)₃ (SEQ ID NO: 15), (Gly₄Gln)₃ (SEQ ID NO: 16), (Gly₃Gln)₄ (SEQ ID NO: 17), (Gly₄Gln)₄ (SEQ ID NO: 18), (Gly₃Gln)₅ (SEQ ID NO: 19), (Gly₄Gln)₅ (SEQ ID NO: 20), (Gly₃Gln)₆ (SEQ ID NO: 21), and (Gly₄Gln)₆ (SEQ ID NO: 22).

17. The multispecific antibody construct of claim 14, wherein the first linker comprises a sequence selected from the group consisting of: (Gly₃Ser)₂ (SEQ ID NO: 1), (Gly₄Ser)₂ (SEQ ID NO: 2), (Gly₃Ser)₃ (SEQ ID NO: 3), (Gly₄Ser)₃ (SEQ ID NO: 4), (Gly₃Ser)₄ (SEQ ID NO: 5), (Gly₄Ser)₄ (SEQ ID NO: 6), (Gly₃Ser)₅ (SEQ ID NO: 7), (Gly₄Ser)₅ (SEQ ID NO: 8), (Gly₃Ser)₆ (SEQ ID NO: 9), and (Gly₄Ser)₆ (SEQ ID NO: 10),

18. The multispecific antibody construct of claim 14, wherein the scFv comprises the first VH attached at its C-terminus to the N-terminus of the first linker and the first linker is attached at its C-terminus to the N-terminus of the first VL.

19. The multispecific antibody construct of claim 14, wherein the scFv comprises the first VL attached at its C-terminus to the N-terminus of the first linker and the first linker is attached at its C-terminus to the N-terminus of the first VH.

20. The multispecific antibody construct of claim 14, wherein the first antigen binding domain and the second antigen binding domain bind to epitopes on different polypeptides.

21. The multispecific antibody construct of claim 14, wherein the first antigen binding domain and the second antigen binding domain bind to different epitopes on the same polypeptide.

22. The multispecific antibody construct of claim 21, wherein the multispecific antibody construct is a biparatopic antibody construct.

23. The multispecific antibody construct of claim 14, wherein the Fc region consists of a hinge region, CH2 region, and CH3 regions.

24. The multispecific antibody construct of claim 14, wherein the N-terminus of the Fc region is linked via its N-terminus to the C-terminus of the heavy chain via a third linker.

25. The multispecific antibody construct of claim 24, wherein the third linker comprises a sequence selected from the group consisting of: (Gly₃Ser)₂ (SEQ ID NO: 1), (Gly₄Ser)₂ (SEQ ID NO: 2), (Gly₃Ser)₃ (SEQ ID NO: 3), (Gly₄Ser)₃ (SEQ ID NO: 4), (Gly₃Ser)₄ (SEQ ID NO: 5), (Gly₄Ser)₄ (SEQ ID NO: 6), (Gly₃Ser)₅ (SEQ ID NO: 7), (Gly₄Ser)₅ (SEQ ID NO: 8), (Gly₃Ser)₆ (SEQ ID NO: 9), (Gly₄Ser)₆ (SEQ ID NO: 10), (Gly₃Gln)₂ (SEQ ID NO: 13), (Gly₄Gln)₂ (SEQ ID NO: 14), (Gly₃ Gln)₃ (SEQ ID NO: 15), (Gly₄Gln)₃ (SEQ ID NO: 16), (Gly₃Gln)₄ (SEQ ID NO: 17), (Gly₄Gln)₄ (SEQ ID NO: 18), (Gly₃Gln)₅ (SEQ ID NO: 19), (Gly₄Gln)₅ (SEQ ID NO: 20), (Gly₃Gln)₆ (SEQ ID NO: 21), and (Gly₄Gln)₆ (SEQ ID NO: 22).

26. The multispecific antibody construct of claim 14, wherein the first polypeptide consists of the antibody Fc region.

27. The multispecific antibody construct of claim 1, wherein the multispecific antibody construct is a bispecific antibody construct.

28. The multispecific antibody construct of claim 1, wherein one CH3 domain comprises a F405L, F405A, F405D, F405E, F405H, F405I, F405K, F405M, F405N, F405Q, F405S, F405T, F405V, F405W, or F405Y mutation; and the other CH3 domain comprises a K409R mutation; wherein the numbering of amino acid residues is according to the EU index as set forth in Kabat.

29. The multispecific antibody construct of claim 1, wherein one CH3 domain comprises a T366W mutation; and the other CH3 domain comprises T366S, L368A, Y407V mutations; wherein the numbering of amino acid residues is according to the EU index as set forth in Kabat.

30. The multispecific antibody construct of claim 1, wherein one CH3 domain comprises K/R409D and K392D mutations; and the other CH3 domain comprises a D399K mutation; wherein the numbering of amino acid residues is according to the EU index as set forth in Kabat.

31. The multispecific antibody construct of claim 30, wherein the CH3 domain which comprises a D399K mutation also comprises a E356K mutation; wherein the numbering of amino acid residues is according to the EU index as set forth in Kabat.
